(19) United States
(12) Patent Application Publication
(10) Pub. No.: US 2019/0321433 A1
(43) Pub. Date: Oct. 24, 2019

(54) PHARMACEUTICAL COMPOSITION AND DIETARY SUPPLEMENT COMPRISING NATURAL EXTRACTS FOR PREVENTING OR TREATING FATTY LIVER DISEASE

(71) Applicant: Bingfang Hu, Cleveland, OH (US)

(72) Inventor: Bingfang Hu, Cleveland, OH (US)

(21) Appl. No.: 15/960,861

(22) Filed: Apr. 24, 2018

(51) Int. Cl. A61K 36/734; A61K 36/537; A61P 1/16; A23L 33/105

(52) U.S. Cl. CPC A61K 36/734; A61K 36/537; A61P 1/16; A23V 2200/32; A61K 2300/00; A23V 2002/00; A23V 2250/21; A23L 33/105

(57) ABSTRACT

The present invention relates to a method for preventing, treating, and ameliorating a fatty liver disease in a person which may be a non-alcoholic liver disease or an alcoholic liver disease in the person. The method comprises administering to the person a therapeutic composition which is a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots, and using the therapeutic composition administered to the person for preventing the fatty liver disease in the person. The ratio of the mixture of the solvent extracts of the Hawthorn fruits and the Salvia miltiorrhiza roots is selected over a range of ratios of between about 0.02 to about 50.

### BACKGROUND OF THE INVENTION

The present invention generally relates to methods for preventing, treating, and ameliorating non-alcoholic fatty liver disease, alcoholic liver disease, and other fatty liver diseases.

Fatty liver disease (FLD) is a reversible condition wherein abnormal retention of lipids and triglycerides accumulate in liver cells via a process of steatosis which is a worldwide disease with multiple causes such as excessive alcohol intake. FLD is observed in up to 80% of obese people. In epidemiological investigations, FLD is recognized as the most common cause of abnormal liver function tests in the United States and occurs in 33% of European-Americans, 45% of Hispanic-Americans, and 24% of African-Americans. Moreover, the prevalence of FLD in the general population ranges from 10% to 24% in various countries.

Based on clinical studies, FLD can be divided into two major categories: alcoholic liver disease (ALD) and non-alcoholic fatty liver disease (NAFLD). Both ALD and NAFLD show microvesicular and macrovesicular fatty changes, but at different stages. NAFLD is related to insulin resistance and metabolic syndrome (obesity, combined hyperlipidemia, diabetes mellitus (type II), and hypertension). The prevalence of NAFLD is between about 12% to 25% in the United States. In severe conditions, the fat accumulation in the liver progressively triggers non-alcoholic steatohepatitis (NASH). For advanced cases of NASH, there are no currently available therapies. Although the exact percentage of patients with NAFLD who go on to develop to liver cancer is not clear, medical research shows there is an association between NAFLD and liver cancer.

ALD encompasses the liver manifestations triggered from excessive ethanol intake, including fatty liver, alcoholic hepatitis, and chronic hepatitis with liver fibrosis or cirrhosis. According to surveys, nearly 80-90% of patients with hepatocellular carcinoma have had ALD. ALD is also the major cause of liver disease in Western countries. According to 2003 data, alcohol-related mortality was the third leading cause of death in United States; and it is still growing rapidly. So far, the foundation of therapy for ALD is alcohol abstinence, nutritional support and corticosteroids treatment. However, patients are often unable to achieve complete and durable abstinence without assistance, and long-term corticosteroids therapy usually leads to multiple adverse reactions.

Facing this serious condition, products have been developed to target FLD. Only a few products have been acceptable to consumers. Silybin, or Silymarin for example, is a natural extract from milk thistle which originated in ancient Greece and Israel. With Silybin, or Silymarin, liver detoxification has been demonstrated in many animal studies, however similar effect in human cases have always ended in conflicting conclusions when based on clinical trials. Furthermore, recent laboratory studies in mice indicate that Silymarin can aggravate alcohol-induced hepatocellular carcinoma in male mice. Thus, there is currently an urgent need for new and improved products to treat FLD.

### BRIEF SUMMARY OF THE INVENTION

In some general embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person.

In other embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person.

In yet other embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

FIG. 1 in accordance with one embodiment of the present invention shows the reversible effect of mixed extracts of Hawthorn fruits and Salvia miltiorrhiza roots towards ethanol diet triggered abnormal liver index. Generally, liver index is the ratio of liver weight divided by body weight. When the ratio of the liver weight divided by body weight is 4% or more in mice, then the liver weight is considered to be abnormal.

FIG. 2 shows results of hematoxylin and eosin (H&E) staining which shows the effect of mixed extracts of Hawthorn fruits and Salvia miltiorrhiza roots in alleviating hepatic balloon-like lesions induced by ethanol diet. Hematoxylin and eosin stain is one of the principal stains in histology. A combination of hematoxylin and eosin, it produces blues, violets, and reds. The Hemalum colors nuclei of cells blue. The eosinophilic structures are generally composed of intracellular or extracellular protein. The Lewy bodies and Mallory bodies are examples of eosinophilic structures. Most of the cytoplasm is eosinophilic. Red blood cells are stained intensely red. The nuclear staining is followed by counterstaining with an aqueous or alcoholic solution of eosin Y, which colors eosinophilic structures in various shades of red, pink and orange.

FIG. 3 shows results of Oil Red O (ORO) which is a stain for fat. ORO demonstrates the beneficial effect of mixed extracts of Hawthorn fruits and Salvia miltiorrhiza roots on inhibiting hepatic lipid accumulation in attenuating hepatic lipid accumulation induced by ethanol diet.

FIG. 4 shows results of serum AST (aspartate aminotransferase) levels and confirms the inhibitory effect of mixed extracts of Hawthorn fruits and Salvia miltiorrhiza roots against the induction of AST triggered by ethanol over-intake.

FIG. 5 shows the reversible effects of mixed extracts of Hawthorn fruits and Salvia miltiorrhiza roots towards exorbitant serum ALT (alanine aminotransferase) levels triggered by ethanol over-intake.

FIG. 6 shows the results of serum triglyceride levels which levels prove the effect of mixed extracts of Hawthorn fruits and Salvia miltiorrhiza roots results in lowering excessive serum TG induction brought by ALD animal model.

FIG. 7 shows results of the inhibitory effect of mixed extract of Hawthorn fruits and Salvia miltiorrhiza roots against the hepatic lipid overloading in ALD (alcoholic liver disease) animal model.

FIG. 8 shows results of mixed extract of Hawthorn fruits and Salvia miltiorrhiza roots in preventing hepatic IL-6 (hepatic interleukin-6) induction triggered by ethanol over-consumption.

FIG. 9 shows the reversible effect of mixed extract of Hawthorn fruits and Salvia miltiorrhiza roots towards an abnormal liver index triggered by an ethanol diet and CCl4 injection (alcoholic liver fibrosis model).

FIG. 10 shows results of H&E staining (hematoxylin and eosin staining) which demonstrates the effect of mixed extract of Hawthorn fruits and Salvia miltiorrhiza roots in alleviating liver fibrosis induced by an alcoholic liver fibrosis model.

FIG. 11 shows results of Oil Red O (ORO) staining for fat. This indicates the effect of mixed extract of Hawthorn fruits and Salvia miltiorrhiza roots in attenuating hepatic lipid accumulation induced by ethanol diet and CCl4 injection.

FIG. 12 shows results of Masson's trichrome staining. Masson's trichrome is a three-color staining protocol used in histology. Masson's trichrome staining recipes produce red keratin and muscle fibers, blue or green collagen and bone, light red or pink cytoplasm, and dark brown to black cell nuclei. In FIG. 12, the Masson's trichrome staining confirmed the inhibitory effect of mixed extract of Hawthorn fruits and Salvia miltiorrhiza roots against formation of fibrotic lesion brought by alcoholic liver fibrosis model.

FIG. 13 shows results of mixed extract of Hawthorn fruits and Salvia miltiorrhiza roots in delaying hasty body weight gain induced by high-fat diet (HFD, obesity and NAFLD animal model).

FIG. 14 shows results of liver index. Generally, liver index is the ratio of liver weight divided by body weight. When the ratio of the liver weight divided by body weight is 4% or more in mice, then the liver weight is considered to be abnormal. The bar graph confirms the inhibitory effect of mixed extract of Hawthorn fruits and Salvia miltiorrhiza roots against abnormal liver index brought by HFD treatment.

FIG. 15 shows results of mixed extract of Hawthorn fruits and Salvia miltiorrhiza roots in alleviating hepatic balloon-like lesion induced by HFD. Which was investigated by H&E staining.

FIG. 16 shows results of Oil Red O (ORO) staining for fat. ORO staining, indicates the effect of mixed extract of Hawthorn fruits and Salvia miltiorrhiza roots in attenuating hepatic lipid accumulation induced by HFD challenge.

FIG. 17 shows results of mixed extract of Hawthorn fruits and Salvia miltiorrhiza roots in preventing induction of serum AST (aspartate aminotransferase) levels triggered by HFD treatment.

FIG. 18 shows the reversible effect of mixed extract of Hawthorn fruits and Salvia miltiorrhiza roots towards exorbitant serum ALT (alanine aminotransferase) levels induced by obesity and NAFLD model.

FIG. 19 shows the results of serum triglyceride levels. Which proved the effect of mixed extract of Hawthorn fruits and Salvia miltiorrhiza roots in lowering excessive serum TG (triglycerides) induction brought by high-fat diet.

FIG. 20 shows the reversible effect of mixed extracts of Hawthorn fruits and Salvia miltiorrhiza roots towards exorbitant serum cholesterol levels triggered by HFD (high fat diet) treatment.

FIG. 21 shows the quantitative analysis of liver lipid accumulation. Which confirmed the inhibitory effect of mixed extracts of Hawthorn fruits and Salvia miltiorrhiza roots against excessive triglyceride increase in mice challenged with NAFLD model.

### DETAILED DESCRIPTION OF THE INVENTION

In general it has been discovered that a composition containing a mixed extract of Hawthorn fruits and Salvia miltiorrhiza roots as an active ingredient is effective in helping to prevent, treat, and ameliorate fatty liver disease; especially non-alcoholic fatty liver disease and alcoholic liver disease. A mixed extract of Hawthorn fruits and Salvia miltiorrhiza roots when used as an active ingredient in a pharmaceutical formulation can help to alleviate fatty liver disease (FLD) by reversing the abnormal liver index, inhibiting hepatic pathogenesis, reducing serum and liver lipid accumulation and decreasing gene expression levels of hepatic pro-inflammatory cytokines. An abnormal liver index is defined to be when a liver has a weight which exceeds 4% of the body weight of a person. As used herein, the term “fatty liver disease” refers to a symptom induced by excessive lipid (i.e., triglycerides [TG]) accumulation in liver. Fatty liver disease can be subdivided into two types: non-alcoholic fatty liver disease (NAFLD) and alcoholic liver disease (ALD), based on pathogenic causes. It is common to observe clinically the following: liver enlargement, abnormal serum aspartate and alanine transaminase levels, exorbitant serum and hepatic cholesterol levels, excessive serum and (TG) liver triglyceride {levels. Specially, NAFLD are usually accompanied with many complications such as, obesity, type II diabetes, insulin resistance and metabolic syndrome. Moreover, ALD may also include several clinical manifestations such as, fever, jaundice, hepatomegaly, hepatic encephalopathy, variceal bleeding, and ascites accumulation.

Some embodiments of the present invention are pharmaceutical compositions, dietary supplements and health functional food compositions which comprise a mixed extract of Hawthorn fruits and Salvia miltiorrhiza roots which are effective when used as an active ingredient for preventing, treating or ameliorating fatty liver disease.

According to the present invention, the mixed extract of Hawthorn fruits and Salvia miltiorrhiza roots have useful pharmacological activities in correcting fatty liver disease (FLD) including the following activities. Mixed extracts of Hawthorn fruits and Salvia miltiorrhiza roots can inhibit hepatic lipid accumulation due to FLD. Mixed extracts of Hawthorn fruits and Salvia miltiorrhiza roots can inhibit an increase in serum aminotransferases due to FLD. Mixed extracts of Hawthorn fruits and Salvia miltiorrhiza roots can inhibit advanced liver tissue pathogenesis due to FLD. Mixed extracts of Hawthorn fruits and Salvia miltiorrhiza roots can inhibit induction of hepatic pro-inflammatory cytokines due to FLD. Mixed extracts of Hawthorn fruits and Salvia miltiorrhiza roots can reverse an abnormal liver index caused by FLD. Note that an abnormal liver index is when the data shows the liver weight exceeds 4% of the body weight.

Based on these experimental results by the Inventor, mixed extracts of Hawthorn fruits and Salvia miltiorrhiza roots can be used as an active ingredient alone or in combination as a component for a pharmaceutical drug, a dietary supplement or a health functional food derived from a natural product. It is conceived that mixed extracts of Hawthorn fruits and Salvia miltiorrhiza roots can be formulated in safe doses that will have a high therapeutic safety index which means that adverse effects to humans can be avoided while providing therapeutic effectiveness for the prevention and treatment of fatty liver disease which may be triggered by obesity and excessive alcohol intake. For the present invention a “person” as claimed includes an animal including a mammal such as an animal or a human being.

For the present invention, the term dosage (or the term dose) is defined herein to mean a weight (for example a mg weight [mg=milligram] of an active ingredient for administration or administered per kilogram weight of the person. The active ingredient is a single chemical substance or a mixture of chemical substances which may be present for example as a solid, or in an extract. The extract may be dry or wet with a liquid, or in an intermediate state such as a paste or a gel or emulsion. Optionally, the active ingredient may be administered in any kind of formulation to the person. For example, a form of the active ingredient may be administered wherein the form is selected from the group consisting of an aerosol suspension of the active ingredient, an aerosol solution of the active ingredient, a liquid solution of the active ingredient, a partial suspension of the active ingredient, a full suspension of the active ingredient, an emulsion of the active ingredient in two liquids, an emulsion of the active ingredient in a liquid, and a gas, a emulsion of the active in a liquid and a mixture of the active ingredient and a food for eating, a mixture of the active ingredient in a liquid for drinking, a suspension of the active ingredient in a solid, a suspension of the active ingredient in a powder, a solid form of the active ingredient by a non-parenteral route of administration, and any combination thereof. Non-parenteral route means a route which for example, can be oral, rectal, nasal, vaginal, dermal, topical, and any other route of administration whereby the active ingredient is not directly delivered as the solid form to the blood circulation of the patient. In addition, for example, the liquid may selected from the group consisting be an alcohol liquid, an alcohol-containing liquid, the alcohol in water, or any liquid non-toxic to a mammalian animal, or any pharmaceutically acceptable liquid and may include a solution such as an isotonic saline.

For the present invention, in defining the term “dosage” or “dose”, the dosage or dose generally means a weight of a “dried material” isolated from the extract administered per kilogram body weight of the person to whom the dose or dosage is being administered. Preferably the dried material may be obtained from an extract comprising an extract of Hawthorn fruits and the Salvia miltiorrhiza roots. The extract is obtained by methods known in the art for obtaining a plant extract. The weight of the dried material of an extract(s) comprises one or more active ingredients, and some of the active ingredients obtainable in an extract of Hawthorn fruits and/or Salvia miltiorrhiza roots have been named by one of their names within the specification of the present patent application. Preferably a dried material form of the active ingredient is weighed by a method known by one of skill in the art of drying an active ingredient obtained from a plant extract (“extract”) or synthetic process for synthesizing the active ingredient(s). For example, a “dried” material form of the active ingredient from the extract can be often be quickly weighed in a tared container on a weighing balance within an analytical balance. The dried material weight may optionally be determined be obtained after some amount of purification or concentration of active ingredients in the extract.

The purification and/or the concentration of the active ingredient can be achieved by any conceivable means such as a known means, a new method, and/or by a means selected from the group consisting of a drying of the extract means, an evaporating liquid in the extract means, a removing liquid from the extract by filtering means, a decanting supernatant liquid after centrifuging the extract means, a separating of two liquid phases means using a separatory funnel with the extract means, a heating of the extract means, a low temperature boiling of the extract means, a vacuum distilling of the extract means, a forming of a salt in the extract means, an adding a salt to the extract means, an adding an acid to the extract means, an adding an alkaline base to the extract means, a changing of the pH of the extract means, a making of an ester in the extract, a drying of a base in the extract means, a chromatography means, an HPLC means, a paper chromatography means, a silica gel chromatography means, a liquid chromatography means, a column chromatography means, and any combination of said means thereof.

For the present invention, the “extract” which contain the active ingredient(s) is obtained by either by obtaining individual extracts or by combining individual extracts. Preferably the extract(s) comprises an extract selected from the group consisting of an extract from the Hawthorn plant, an extract from Hawthorn fruits, an extract from the Salvia miltiorrhiza plant, an extract from Salvia miltorrhiza roots and/or a combination thereof. A preferred dosage or dose may be administered as the extract. Once the concentration of the dried material is known in the extract when the extract is in a liquid form, then a volume of the extract can be usefully known to have a weight of the dried material per liquid volume of the extract. A volume of the extract then can be used as the dose or dosage per kilogram weight of the person to whom the dose or dosage is bing administered. A preferred extract for the present invention is a mixed extract of the Hawthorn fruits and the extract Salvia miltiorrhiza roots. Another preferred extract for the present invention is either an extract of the hawthorn fruits or/and an extract of the Salvia miltiorrhiza roots.

Preferably the extract is dried to obtain the dried material. The weight of the dried material is preferably obtained by weighing the dried material with the active ingredients in a preferred physical state. For example, the dried material may comprise one or more active ingredients in a preferred state selected from the group consisting of a crystalline state, an amorphous state, a powder state, a lumpy state, a flaky state, a syrup-like substance state, an oily substance state, a resinous state, and a combination thereof. Less preferred is when the dried material is tarry or gummy or pasty. However, the practicing of the methods of the present invention is not intended to be restricted by the difficulties of obtaining a precise weight of the active ingredient in the extract. In addition, the active ingredients should not be carelessly or accidentally caused to become damaged from sunlight, other forms of electromagnetic radiation such as heat, microwaves, or ultraviolet light.

For the present invention, methods of preventing a fatty liver disease in a person are a means for keeping fatty liver disease from happening or arising in the person. For the present invention, methods of treating a fatty liver disease in a person are a means for keeping a fatty liver disease from happening or arising in the person. For the present invention, methods of ameliorating a fatty liver disease in a person are means for keeping a fatty liver disease from happening or arising in the person.

For the present invention a fatty liver is defined as follows. Fatty liver may be diagnosed in the asymptomatic patient who is undergoing evaluation for abnormal liver function tests; and aminotransferase levels may less than twice the upper limit of normal. No laboratory test alone is an absolute diagnostic of fatty liver. Characteristic ultrasonographic findings of fatty liver disease include a hyperechoic liver with or without hepatomegaly. Liver biopsy is rarely needed to diagnose fatty liver in the appropriate clinical setting, but it may be useful in excluding steatohepatitis or fibrosis. Typical histologic findings of fatty liver can include fat accumulation in hepatocytes that is often macrovesicular and occasionally microvesicular. The centrilobular region of the hepatic acinus can be affected. In severe fatty liver, fat (triglycerides [TG] are a type of fat) is distributed throughout the acinus. Fatty liver can have various causes. Attribution of a fatty liver to alcohol use can require a detailed and accurate patient history.

For the present invention, a diagnosis of alcoholic hepatitis can be based on a thorough history, physical examination, and review of laboratory tests. The ratio of aspartate aminotransferase (AST) to alanine aminotransferase (ALT) can become about 2:1 and the absolute aminotransferase level usually does not exceed ˜300 U/L unless there is also a superimposed hepatic insult exists, such as acetaminophen toxicity. Other common and nonspecific laboratory abnormalities may include anemia and leukocytosis. A liver biopsy may be needed to become sure of the diagnosis.

Classic histologic features of alcoholic hepatitis include inflammation and necrosis, which are most prominent in the centrilobular region of the hepatic acinus. Hepatocytes are classically ballooned, which causes compression of the sinusoid and reversible portal hypertension. The inflammatory cell infiltrate, located primarily in the sinusoids and close to necrotic hepatocytes, consists of polymorphonuclear cells and mononuclear cells. In addition to inflammation and necrosis, a patient with alcoholic hepatitis can have fatty infiltration and Mallory bodies, which are intracellular perinuclear aggregations of intermediate filaments that are eosinophilic on hematoxylin-eosin staining. Neither fatty infiltration nor Mallory bodies may be specific for alcoholic hepatitis or needed to be discovered to establish the diagnosis.

The diagnosis of alcoholic cirrhosis includes finding classic signs and symptoms of end-stage liver disease in a patient with a history of significant alcohol intake. Patients tend to underreport their alcohol consumption, and discussions with family members and close friends can be needed to accurately estimate the person's alcohol intake.

Alcoholic cirrhosis patients can have complications of portal hypertension, including ascites, variceal bleeding, and hepatic encephalopathy. The histology of end-stage alcoholic cirrhosis, in the absence of acute alcoholic hepatitis, resembles that of advanced liver disease from many other causes. The methods for the clinical diagnosis of alcoholic liver disease, using a combination of physical findings, laboratory values, and clinical acumen, is well established but may include a liver biopsy. Methods of the present invention are useful, and novel methods for preventing, treating, and ameliorating non-alcoholic fatty liver disease, alcoholic liver disease, and other fatty liver diseases.

1. In some general embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person.

2. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the mixture of the pharmaceutically acceptable solvent extracts of the Hawthorn fruits and the Salvia miltiorrhiza roots is ethyl alcohol solvent extracts of the Hawthorn fruits and the Salvia miltiorrhiza roots.

3. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the pharmaceutically acceptable solvent extracts of the Hawthorn fruits and the Salvia miltiorrhiza roots are ethyl alcohol in water solvent extracts of the Hawthorn fruits and the Salvia miltiorrhiza roots.

4. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the fatty liver disease is selected from the group consisting of a non-alcoholic fatty liver disease, and an alcoholic liver disease.

5. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the mixture of the ethyl alcohol solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

6. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the mixture of the ethyl alcohol in the water solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

7. In some embodiments, the invention is a method for preventing a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for preventing the fatty liver disease in the person,

wherein the therapeutic composition further comprises an excipient ingredient selected from the group consisting of a pharmaceutically acceptable carrier, a purified water, an isotonic saline, a surfactant, a flavoring agent, a colorant, a diluent, a disintegrant, a sugar, a protein, a fat, a gum, and any combination thereof,

wherein the administering to the person of the therapeutic composition occurs by a route of administration selected from the group consisting of an oral route, a parenteral route, an intravenous route, a subcutaneous route, an intraperitoneal route, a topical application, a transdermal patch route, a nasal spray route, a pulmonary aerosol route, a suppository route, and any combination thereof, and

wherein a total daily dosage of the mixture of pharmaceutically acceptable solvent extracts of the Hawthorn fruits and the Salvia miltiorrhiza roots as calculated per kilogram body weight of the person is the total daily dosage selected from the group consisting of between about 1 mg to about 10 mg, between about 10 mg to about 40 mg, between about 40 mg to about 70 mg, between about 70 mg to about 100 mg, between about 100 mg to about 130 mg, between about 130 mg to about 160 mg, between about 160 mg to about 190 mg, between about 190 mg to about 210 mg, between about 210 mg to about 240 mg, between about 240 mg to about 270 mg, between about 270 mg to about 300 mg, between about 300 mg to about 330 mg, between about 330 mg to about 360 mg, between about 360 mg to about 390 mg, between about 390 mg to about 420 mg, and between about 420 mg to about 450 mg, and

wherein the total daily dosage of the mixture of pharmaceutically acceptable solvent extracts of the Hawthorn fruits and the Salvia miltiorrhiza roots is administered in one dose per day or in divided doses each day.

8. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person.

9. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the mixture of the pharmaceutically acceptable solvent extracts of the Hawthorn fruits and the Salvia miltiorrhiza roots are ethyl alcohol solvent extracts of the Hawthorn fruits and the Salvia miltiorrhiza roots.

10. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the mixture of the pharmaceutically acceptable solvent extracts of the Hawthorn fruits and the Salvia miltiorrhiza roots are ethyl alcohol in water solvent extracts of the Hawthorn fruits and the Salvia miltiorrhiza roots.

11. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the fatty liver disease is selected from the group consisting of a non-alcoholic fatty liver disease, and an alcoholic fatty liver disease.

12. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the mixture of the ethyl alcohol solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

13. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the mixture of the ethyl alcohol in the water solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

14. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the therapeutic composition is the health functional food composition, wherein the mixture of the pharmaceutically acceptable solvent extracts of the Hawthorn fruits and the Salvia miltiorrhiza roots is added to the therapeutic health food composition, and

wherein the mixture of the pharmaceutically acceptable solvent extracts of the Hawthorn fruits and the Salvia miltiorrhiza roots has a weight which is between about 0.1 percent to about 12 percent of the weight of the therapeutic health food composition.

15. In some embodiments, the invention is a method for treating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for treating the fatty liver disease in the person,

wherein the therapeutic composition further comprises an excipient ingredient selected from the group consisting of a pharmaceutically acceptable carrier, a purified water, an isotonic saline, a surfactant, a flavoring agent, a colorant, a diluent, a disintegrant, a sugar, a protein, a fat, a gum, and any combination thereof,

wherein the therapeutic composition is administered by a route of administration selected from the group consisting of an oral route, a parenteral route, an intravenous route, a subcutaneous route, an intraperitoneal route, a topical application, a transdermal patch route, a nasal spray route, a pulmonary aerosol route, a rectal suppository route, and any combination thereof, and

wherein a total daily dosage of the mixture of pharmaceutically acceptable solvent extracts of the Hawthorn fruits and the Salvia miltiorrhiza roots as calculated per kilogram body weight of the person is selected from the group consisting of between about 1 mg to about 10 mg, between about 10 mg to about 40 mg, between about 40 mg to about 70 mg, between about 70 mg to about 100 mg, between about 100 mg to about 130 mg, between about 130 mg to about 160 mg, between about 160 mg to about 190 mg, between about 190 mg to about 210 mg, between about 210 mg to about 240 mg, between about 240 mg to about 270 mg, between about 270 mg to about 300 mg, between about 300 mg to about 330 mg, between about 330 mg to about 360 mg, between about 360 mg to about 390 mg, between about 390 mg to about 420 mg, and between about 420 mg to about 450 mg, and

wherein the total daily dosage of the mixture of pharmaceutically acceptable solvent extracts of the Hawthorn fruits and the Salvia miltiorrhiza roots is administered in one dose a day, or in divided doses during the day.

16. In some embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person.

17. In some embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person,

wherein the mixture of the pharmaceutically acceptable solvent extracts of the Hawthorn fruits and the Salvia miltiorrhiza roots are ethyl alcohol solvent extracts of the Hawthorn fruits and the Salvia miltiorrhiza roots.

18. In some embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person,

wherein the mixture of the pharmaceutically acceptable solvent extracts of the Hawthorn fruits and the Salvia miltiorrhiza roots are ethyl alcohol in water solvent extracts of the Hawthorn fruits and the Salvia miltiorrhiza roots.

19. In some embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person,

wherein the mixture of the pharmaceutically acceptable solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, between about 10 to about 50.

20. In some embodiments, the invention is a method for ameliorating a fatty liver disease in a person, the method comprising: administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and using the therapeutic composition administered to the person for ameliorating the fatty liver disease in the person,

wherein the fatty liver disease is selected from the group consisting of an alcoholic liver disease, and a non-alcoholic fatty liver disease.

Additionally, the Hawthorn fruits extract may contain a composition of Hawthorn fruit flavonoids as pharmacological ingredients. Hawthorn fruits flavonoids can be used to lower blood pressure and attenuate cardiovascular pathogenesis. Preferably, the Hawthorn fruits flavonoids contain Rutin, Hyperoside and Vitexin. Preferably, a Hawthorn fruits extract should contain no less than 10%, flavonoids when the Hawthorn fruits extract is used in a dose for preventing or treating fatty liver disease,

In some embodiments, the present invention relates to various compositions and various therapeutic uses of a therapeutic chemical compound obtained from a botanical source as an extract or obtained from a synthetic organic chemistry source. A synthetic therapeutic chemical compound may be obtained by using one or more organic chemical synthesis process steps using one or more known organic chemical methods and using one or more intermediate or starting organic chemical compounds, reagents, catalysts, chiral reagents, chiral intermediates, blocking groups, solvents, and energy sources. Alternatively, the therapeutic chemical compound may be purchased as a pure chemical compound or an impure chemical compound mixture, or may be obtained from a plant source.

One of skill in the art of extraction of therapeutic chemical substances from a botanical source can obtain a therapeutic chemical compound from a plant by various methods to make an extract containing the therapeutic chemical compound from the plant. A part of a plant from which to obtain the therapeutic chemical compound, may include a plant part material selected from the group consisting of a root, a fruit, a seed, a flower, a stem, a leaf, a tuber, a bulb, a bark, a twig, a xylem, a phloem, and a vegetable. A plant extract is a substance made by extracting a part of a raw material of the plant, often by using a solvent such as ethanol or water. One kind of a plant extract is a tincture. In a tincture, the therapeutic chemical compound extracted from the plant is in an alcohol. A second kind of a plant extract is absolutes. In absolutes, the therapeutic chemical compound is extracted from the plant using a solvents including hexane and after all solvents have been removed, what remains is a highly concentrated oily form of the therapeutic chemical compound. Absolutes are a typical form of a highly aromatic oil. A third kind of a plant extract is a powder. In a powder, the therapeutic chemical compound is extracted from the plant using solvents so that only the insoluble plant fiber is not dissolved. The therapeutic chemical compound is present with other solvent-solubilized substances as a solute in the solvent. When the solvent is removed by evaporation, vacuum distillation or some other process, the solute then appears a powder. For the solute in the solvent to become a powder the solute must not contain a gum, or be so impure that the solvent becomes trapped in the solids, or the solids are over-heated in the process of drying and burn and form a tarry solid.

An effective and efficient method for obtaining a plant extract depends upon selecting a suitable liquid solvent which functions as a selective solvent for extract formation. This means the desired therapeutic chemical compound from the plant material has a higher solubility in the selective solvent that the undesired chemical substance from the plant material. The plant material is repetitively extracted with aliquots of selective solvent. The mixture is filtered is there is simply an extraction of plant material by a selective solvent. If there are multiple solvents which are immiscible to some extent, then the mixture of the plant material and two liquid solvents use a separatory funnel (“sep funnel”). One of the solvents is the selective solvent. Typically as the two immiscible liquids separate, an emulsion forms. The lesser dense liquid floats on top of the denser liquid. The clean part of the selective solvent fractions is set aside and saved. The plant material is usually then again extracted again with a fresh volume of the selective solvent. Each time the selective solvent is recovered and saved. This extraction process can be repeated several times until almost all of the desired therapeutic chemical substance has been solubilized into the selective solvent fractions that have been set aside and saved. The therapeutic chemical compound in the selective solvent constitutes an extract from the plant material. The therapeutic chemical compound is a solute in the selective solvent. The extract can be concentrated by evaporating away some of the selective solvent in the extract. Solvent evaporation of organic compounds is usually done at a temperature below the temperature where the therapeutic chemical compound is volatile and could be lost to the atmosphere. Solvent can be often safely removed by on for example by passive evaporation, or by distillation under a vacuum, or by a freeze drying process.

Preferably conditions for obtaining the extract are carefully selected to optimally obtain a preferred composition in terms of yield of extract initially followed by a purification of the extract to remove undesirable chemicals that ended up in the extract. Many conditions can be adjusted to optimize an extraction process. The extraction conditions include, the weight amount of the plant to the volume of the selective solvent. The extraction temperature, mixing rate, and mixing time. In a liquid-liquid extraction of the therapeutic chemical compound in a separatory funnel system, the therapeutic chemical compound may become ionized depending upon the pH of the extraction mixture. The extraction mixture pH can be altered to select a pH where the therapeutic chemical compound is ionized so that it is more water soluble. Alternatively, the extraction mixture pH can be altered to select a pH where the therapeutic chemical compound is not ionized and is more soluble in an organic solvent phase than in a water phase. With such therapeutic chemical compounds it is possible to both concentrate and purify them selectively by choosing the optimal pair of immiscible solvents, temperatures, and pH conditions.

Other factors in the extraction yield and possible purity of the isolated therapeutic chemical compound will include the freshness or spoilage of the plant after its picking from the ground where it was grown, the maceration state of the plant, the dryness of the plant, and plant contamination by exogenous substances not expected to be present. Often a dried part of a plant is preferred for extraction. Dried plant material crumble easily to form small fragments with a high surface area which facilitates the extraction process.

Solubility is the property of a solid, liquid, or gaseous chemical substance called solute to dissolve in a solid, liquid, or gaseous solvent. The solubility of a substance fundamentally depends on the physical and chemical properties of the solute and solvent as well as on temperature, pressure and the pH of the solution. The extent of the solubility of a substance in a specific solvent is measured as the saturation concentration, where adding more solute does not increase the concentration of the solution and begins to precipitate the excess amount of solute. Most often, the solvent is a liquid, which can be a pure substance or a mixture of different liquid solvents. Under certain conditions, the equilibrium solubility can be exceeded to give a so-called supersaturated solution, which is metastable. Meta-stability of crystals can also lead to apparent differences in the amount of a chemical that dissolves and depends on crystalline form, polymorphisms, and particle size. A supersaturated solution generally crystallizes when ‘seed’ crystals are introduced and rapid equilibration occurs. Solubility of a substance is a different property from the rate of solution formation which increases the smaller particle is.

The solubility term to be used for the therapeutic chemical compound(s) of the present invention may include solubility terms agreed to by IUPAC (International Union of Pure and Applied Chemistry) According to the IUPAC definitions, solubility is the analytical composition of a saturated solution expressed as a proportion of a designated solute in a designated solvent. Solubility may be stated in various units of concentration such as molarity, molality, mole fraction, mole ratio, mass (solute) per volume(solvent) and other units. The extent of solubility of one substance with another substance ranges widely. There are infinitely soluble liquids which are termed fully miscible such as ethanol in water. There are poorly soluble solutes such as the compound silver chloride in water as the solvent. The term insoluble is often used to describe a poorly or very poorly soluble compounds. The USP (U.S. Pharmacopoeia) has seven qualitative labels to use to describe the solubility of a solute in a particular solvent based on the mass parts of the particular solvent that are required to dissolve one mass part of the solute.

(1) A very soluble solute in a solvent means that less than 1 mass part of the solvent is required to dissolve one mass part of the solute.
(2) A freely soluble solute in a solvent means that 1 to 10 mass parts of the solvent is required to dissolve one mass part of the solute.
(3) Soluble solute in a solvent means that 10 to 30 mass parts of the solvent is required to dissolve one mass part of the solute.
(4) Sparingly soluble solute in a solvent means that 30 to 100 mass parts of the solvent is required to dissolve one mass part of the solute.
(5) Slightly soluble solute in a solvent means that 100 to 1000 mass parts of the solvent is required to dissolve one mass part of the solute.
(6) Very slightly soluble solute in a solvent means that 1000 to 10,000 mass parts of the solvent is required to dissolve one mass part of the solute.
(7) Practically insoluble or insoluble solute in a solvent means than greater than 10,000 mass parts of the solvent is required to dissolve one mass part of the solute.

The present invention provides a pharmaceutical composition for prevention or treatment of fatty liver disease, especially NAFLD and ALD, containing a mixed extract of Hawthorn fruits and Salvia miltiorrhiza roots as an active ingredient. Additionally, the composition may further contain an extract(s) of other herbs which have been proved for the hepatoprotective effect. The mixed extract between the Hawthorn fruits extract and Salvia miltiorrhiza roots extract may be prepared by mixing them at a ratio of 2:1 to 1:2, or prepared by mixing the Hawthorn fruits and Salvia miltiorrhiza roots at a ratio of 2:1 to 1:2, followed by extraction, but is not limited thereto.

For some embodiments of the present invention, therapeutic chemical compounds are obtained from solvent extractions of parts of plant material from Salvia miltiorrhiza plant species. The Salvia miltiorrhiza plant extracts will contain one or more therapeutic chemical compounds. Some of the chemical compounds that may be extracted from a Salvia miltiorrhiza plant species have a known molecular structure and name. Specific therapeutic chemical compounds described in the present specification are identified as found in extracts of Salvia miltiorrhiza plant species. The plant Salvia miltiorrhiza, is also known as red sage, Chinese sage, tan shen, or danshen. Danshen is a perennial plant that grows in China and in Japan at elevations of between 300 to 3,940 ft above sea level, and at those altitudes in grassy places in forests, on hillsides, and along stream banks.

Salvia miltiorrhiza roots extract can contain a series of total Tanshinone as pharmacological ingredients. Preferably, the Tanshinone mixture contain Tanshinone IIA, Tanshinone I, Tanshinone IIB, Cryptotanshinone and Dihydrotanshinone. To have ideal performance in preventing or treating fatty liver disease, a Salvia miltiorrhiza roots extract should contain not less than 10% of the total tanshinone. Cryptotanshinone (CTS), tanshinone I (TSI) and tanshinone IIA (TSA) are the major lipophilic compounds that may be obtained in an extract of Salvia miltiorrhiza. The molecular structure for CTS, TSI, and TSA compounds comprises a diterpenoidal quinone structure (LuXing, 2017). An extract from the plant Salvia miltiorrhiza which contains an amount of a diterpenoidal quinone may be obtained by a liquid extraction of the dried or fresh plant Salvia miltiorrhiza using ethyl alcohol as the extraction solvent alone or in combination with water. An alcohol solvent may be selected from the group consisting of an organic solvent, water, water and an organic solvent, a mixture of organic solvents, a mixture of organic solvents and water, and a combination thereof. For example, preferably a mixture of an alcohol and water is used. Preferably, a Salvia miltiorrhiza roots extract is prepared by extracting using water, methanol, ethanol, butanol, or a mixed solvent thereof, but is not limited thereto. Also, the alcohol may be a C₁-C₁₀ alcohol selected from the group consisting of a C₁-C₁₀ primary alcohol, a C₁-C₁₀ secondary, a C₁-C₁₀ tertiary alcohol, and any combination thereof. Specific examples of alcohols that may be used to a make a Salvia miltiorrhiza roots extract include methanol, ethanol, 1-propanol, 2-propanol, 1-butanol, 2-butanol, tert-butanol, 1-pentanol, 2-pentanol, 3-pentanol, 1-hexanol, cyclopentanol, cyclohexanol, heptanol, octanol, nonanol, decanol, phenol, and benzyl alcohol. Methyl alcohol, ethyl alcohol, propyl alcohol, and isopropyl alcohol are highly soluble in water and thus mixtures of these alcohols I water are particularly useful for making a Salvia miltiorrhiza roots extract.

Also, there are phenolic water-soluble compounds which may be extracted from roots of Salvia miltiorrhiza, preferably dried roots of Salvia miltiorrhiza. These 6 phenolic compounds are (a) magnesium lithospermate B (MLB), (b) rosmarinic acid (RA), (c) lithospermic acid (LA), (d) caffeic acid (CAA), (e) protocatechuric aldehyde (3,4-dihydroxybenzaldehyde, PAI), and (f) 3,4-dihydroxyphenyllactic acid (danshensu).

Specific therapeutic chemical compounds described in the present specification are identified as found in extracts of the hawthorn plant species Crataegus oxyacantha and other Crataegus species. There are more than 300 species of the hawthorn plant throughout the world. The hawthorn plant is a dense, thorny shrub that grows 5-13 ft (1.5-4 m) high and has white flowers that look like roses that flower in the spring. Hawthorn grows throughout the world anywhere that is moist. Extracts are made from the hawthorn plant's flowers, leaves, and berries. The hawthorn berries are the hawthorn fruit. The hawthorn also is called Crataegus extract, mayflower, maybush, and whitethorn. Common trade names for hawthorn include Cardiplant, Hawthorn Berry, Hawthorn Formula, Hawthorn Heart, Hawthorn Phytosome, and Hawthorn Power.

As used herein, the term “a mixed extract of Hawthorn fruits and Salvia miltiorrhiza roots” refers to a mixture between a Hawthorn fruit extract and a Salvia miltiorrhiza root extract; or an extract prepared by extracting the mixture of Hawthorn fruits and Salvia miltiorrhiza roots. “Hawthorn fruit” is known to have effects of antioxidation, alleviating cardiovascular disease and improving exercise tolerance. Preferably, a Hawthorn fruits extract is prepared by extracting using water, methanol, ethanol, butanol, or a mixed solvent thereof, but is not limited thereto.

An extract of Hawthorn fruits and an extract of Salvia miltiorrhiza roots may be obtained separately or at the same time by fully mixing the herb materials with ethanol or another alcohol, or an alcohol with water followed by extraction step. The process may be performed via room temperature extraction, hot water extraction, cold immersion extraction, reflux cooling extraction, ultra-sonification extraction, supercritical extraction, or vapor extraction, but is not limited thereto. Additionally, the primary extract can then be refined by purification process to obtain a extract with more flavonoids content.

In the present invention, it was confirmed that a composition containing a mixed extract of Hawthorn fruits and Salvia miltiorrhiza roots is effective for preventing and treating fatty liver disease. In some embodiments of the present invention, the mixed extract of Hawthorn fruits and Salvia miltiorrhiza roots usefully inhibits the pathogenesis brought by high-fat induced NAFLD/obesity, alcoholic over-intake induced hepatosteatosis, and alcohol-Carbon tetrachloride induced liver fibrosis. More specifically, the mixed extract largely reversed the abnormal liver index, lipid accumulations and histological lesion. In addition, the mixed extracts of Hawthorn fruits and Salvia miltiorrhiza roots dramatically decreased induction of hepatic pro-inflammatory cytokines, serum aminotransferase and triglyceride levels. Additionally, the mixed extract was also shown to delay sudden body weight gain caused by a high fat-diet. The Inventor's experimental results are consistent in showing the usefulness of the mixed extract of Hawthorn fruits and Salvia miltiorrhiza roots for the prevention and treatment of fatty liver disease and there is conceived to be a great potentiality for developing pharmaceutical compositions, dietary supplements and health functional food compositions which comprise the mixed extract(s) of Hawthorn fruits and Salvia miltiorrhiza roots.

The composition of the present invention may include a pharmaceutically acceptable carrier, an excipient, or a diluent, in addition to the active ingredients described above, for administration purposes. The composition of the present invention may be formulated in the form of an oral administration such as powders, granules, tablets, capsules, suspensions, emulsions, syrups, and aerosols; external applications, suppositories, and sterile injection solutions, according to the respective conventional method to be used.

The composition of the present invention, according to the desired purpose, may be administered orally or parenterally (e.g. intravenous, subcutaneous, intraperitoneal, or topical application). The dosage may vary according to the health status, body weight, severity of a disease of a patient, drug types, administration routes, and duration, but they may be appropriately selected by one of ordinary skill in the art.

The daily dosage of the mixed extract of Hawthorn fruits and Salvia miltiorrhiza roots is preferably in the range of 5 mg/kg to 150 mg/kg, and may be administered in a few divided doses, as necessary.

Additionally, the present invention provides a dietary supplement/health functional food composition for preventing or ameliorating (improving) fatty liver disease containing a mixed extract of Hawthorn fruits and Salvia miltiorrhiza roots as an active ingredient.

Preferably, the dietary supplement, or the health functional food composition may further contain a food additive, and its acceptability as a “food additive” should be determined based on the standards and criteria regarding the corresponding items according to the General Provisions and General Test Methods of FDA, unless specified otherwise. In particular, the amount of the mixed extract to be added to the foods including beverages during the manufacture of the health functional food may be appropriately added or reduced as necessary, and preferably, these extracts may be added in the range of 0.1 wt % to 12 wt % relative to 100 wt % of the food.

A better understanding of the present invention may be obtained through the following Examples and Experimental Examples. However, they are disclosed for illustrative purposes only, and should not be construed as limiting the scope of the present invention.

### Experiment Example 1

The inhibitory effect of mixed extract against ethanol diet induced Alcoholic hepatosteatosis. Experiments were performed to examine the beneficial effects of mixed extract, as well as each compositions in treating ethanol diet induced mice hepatosteatosis.

Extracts Preparation—

Ethanol extracts of Hawthorn fruits containing different proportion of flavonoid contents (3-99%) are all commercially available. Additionally, extracts from Salvia miltiorrhiza roots containing diverse proportion of tanshinone contents (3-99%) can also be purchased. In the present invention, Hawthorn fruits extract (containing 80% flavonoid) and Salvia miltiorrhiza roots extract (containing 40% tanshinone) were purchased separately and thoroughly mixed to obtain selected weight ratios. For convenience, the dosages mentioned below represent flavonoid contents in Hawthorn fruits extract, or tanshinone contents in Salvia miltiorrhiza roots extract. Which were commonly accepted most active ingredients in the ethanol extract of both herbs. The weight ratio in mixed extract is 5:4 (Hawthorn fruits flavonoid:tanshinone).

Experimental Animal—

This experiment strictly followed the ALD (Alcoholic Liver Disease) model released by NIAAA (National Institute on Alcohol Abuse and Alcoholism). After 5 days of a liquid control diet (Lieber-DeCarli diet ad libitum) adaption, C57BU6 mice (8-9 weeks, male) were then randomly divided into 7 experimental animal groups.

### Experimental Animal Groups:

1 Control diet group,

2. Ethanol diet-vehicle group (V),

3 Ethanol diet-Hawthorn fruits extract group (H),

4 Ethanol diet-Salvia miltiorrhiza roots (S) extract group and

5 Ethanol diet-mixed extract groups at low dosage (M-L)

6 Ethanol diet-mixed extract groups at low dosage (M-M)

7 Ethanol diet-mixed extract groups at high dosages (M-H).

Briefly, the ALD model was conduct by challenging mice with liquid ethanol diet (Lieber-DeCarli diet ad libitum with 5% ethanol (v/v)) for 10 consecutive days. Nine hours before tissue harvesting, mice were orally gavaged with ethanol at dosage of 5 g/kg. For extract groups, mice were daily treated with either Hawthorn fruits extract (150 mg/kg), Salvia miltiorrhiza roots extract (120 mg/kg) alone or mixed extracts (H-S: 75-60, 150-120, 300-240 mg/kg) intra-gastrically, along with the ethanol diet. The vehicle group was administered with corresponding solvent. The control group was fed with control diet during the whole model.

Statistical Analysis—

The comparison between two groups was analyzed by student T test. The symbol “NS” is used to means the students T test found no statistically significant difference (P value set to less than 0.05 for statistical significance). Note that P values which are *P<0.05, **P<0.01, ***P<0.001, #P<0.05, ##P<0.01, ※P<0.05, ※※※P<0.001 all represent significant differences between two comparison groups.

### Experiment Example 1 Results

According to macroscopic observation, the livers in mice challenged with ALD model were significantly larger than control group.

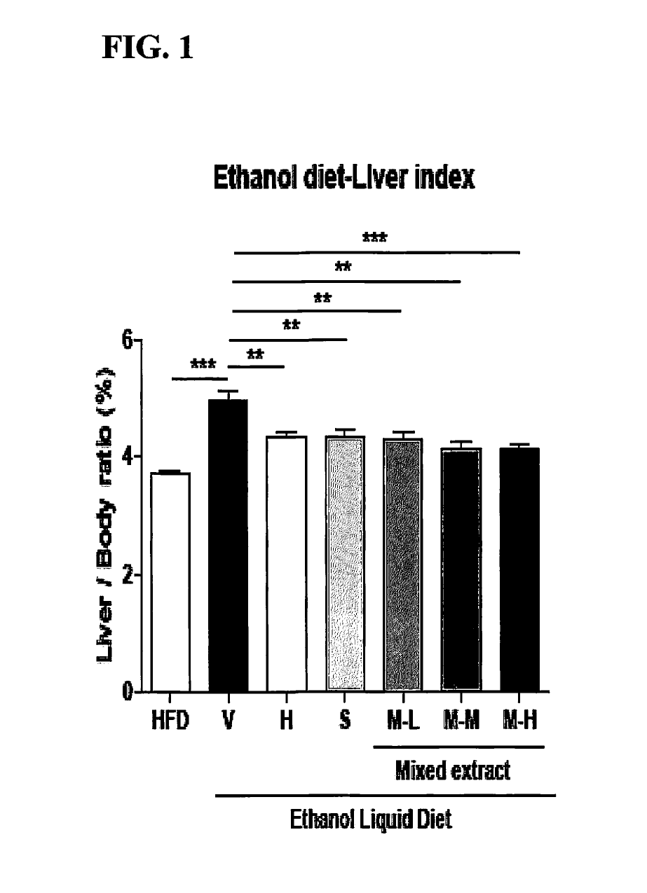

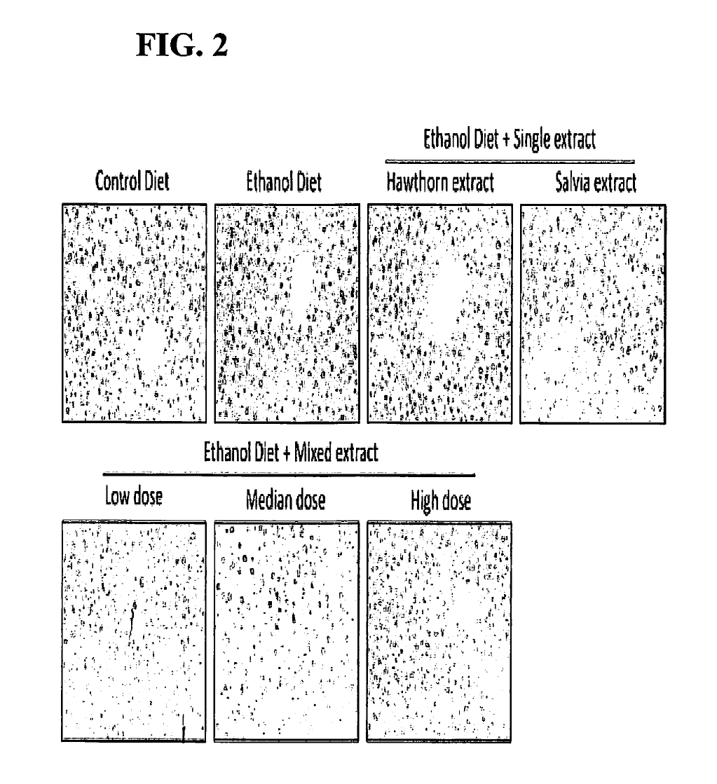

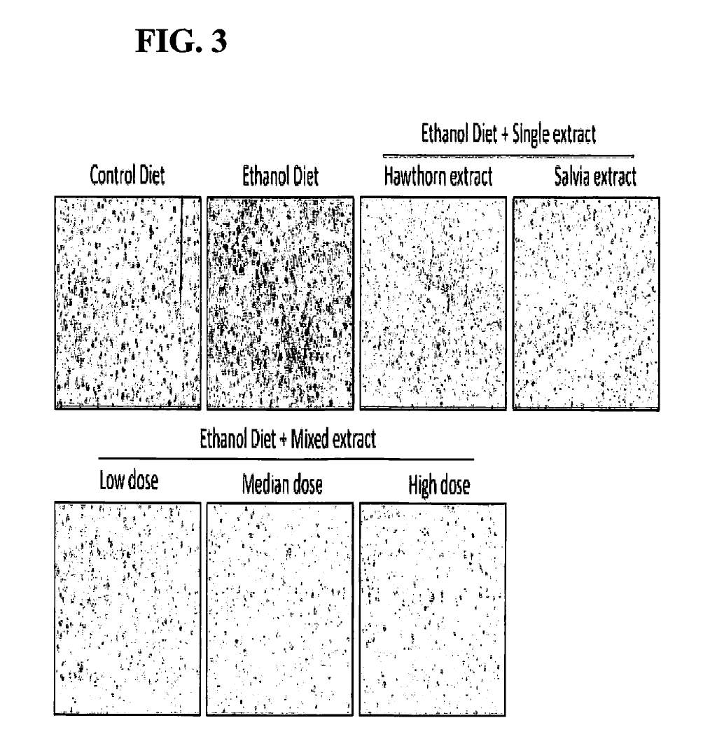

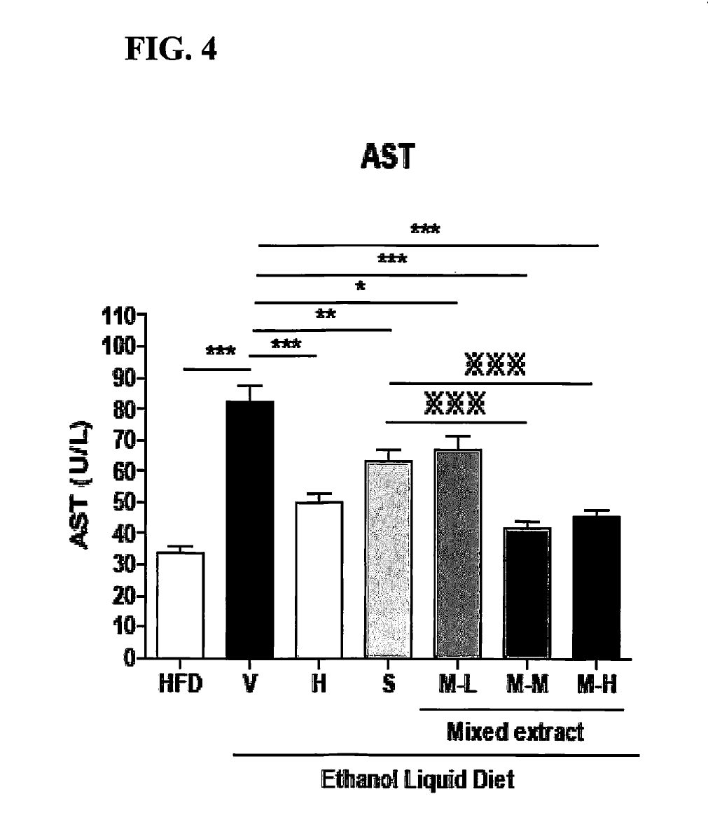

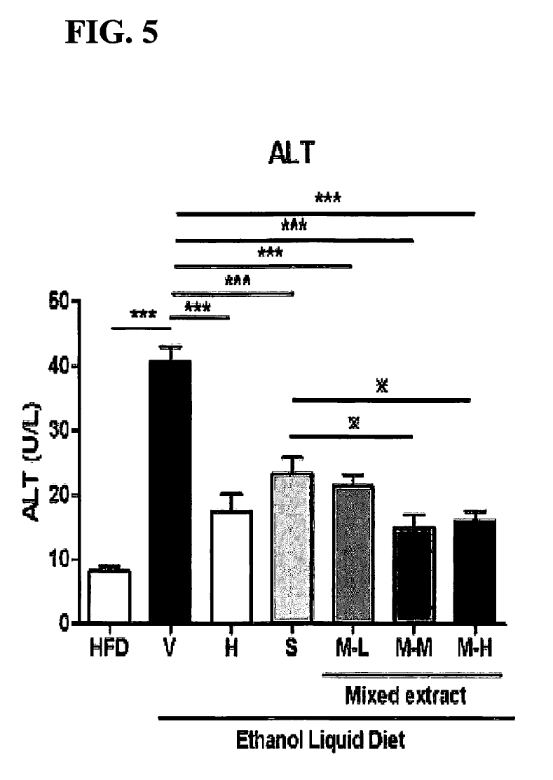

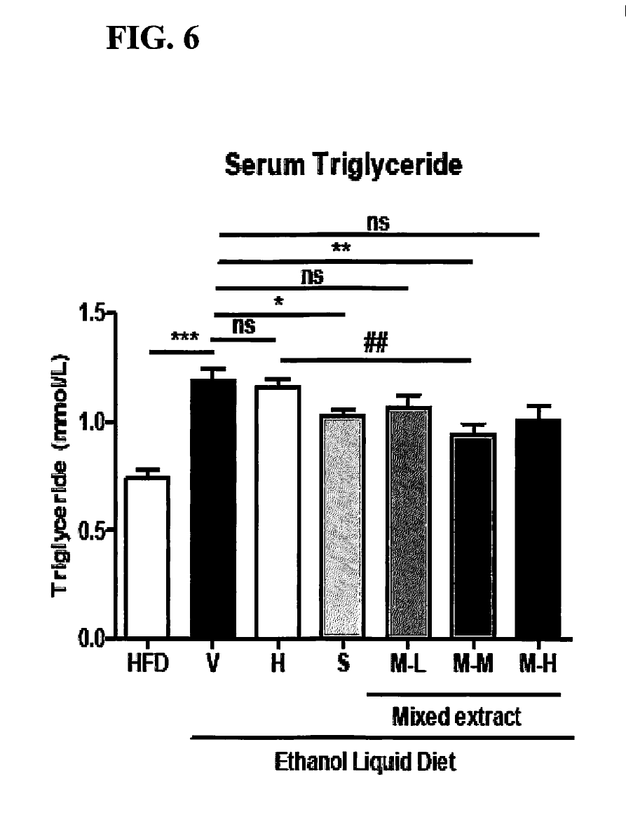

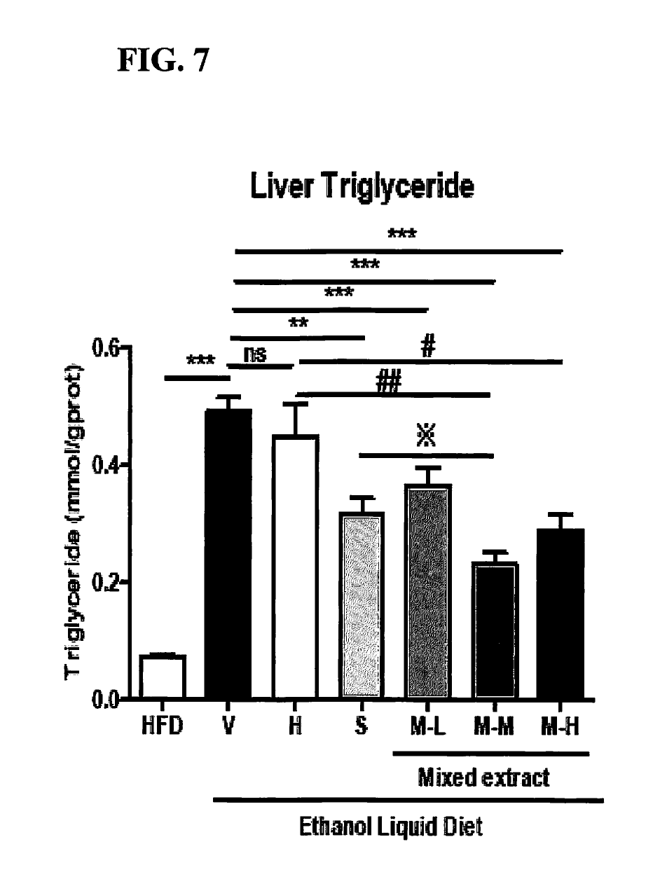

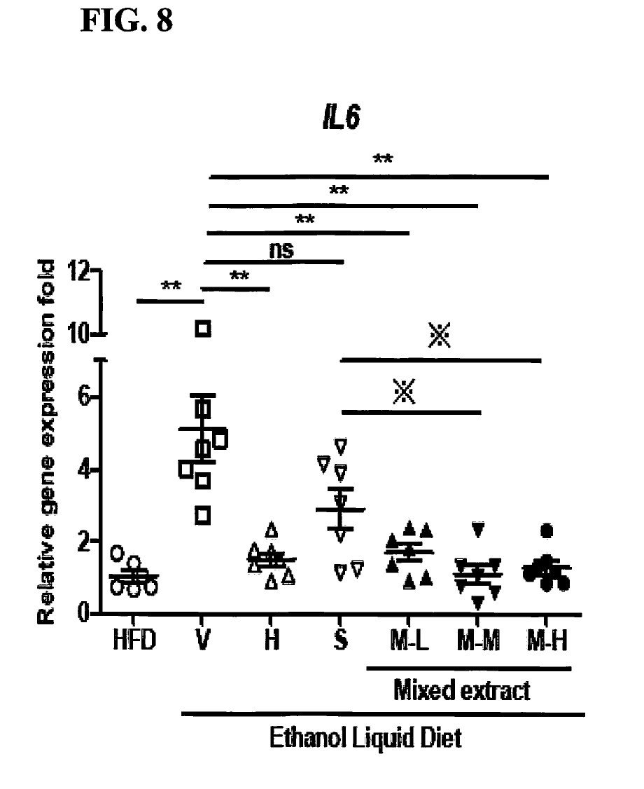

Quantitative analysis also confirmed that the mice with ethanol over-intake had heavier liver and abnormal liver index. Extract treatments largely attenuated this condition See FIG. 1. Moreover, histological analysis also demonstrated the hepatic pathogenesis in ALD mice. Both H&E staining and ORO staining detected the balloon-like lesion and lipid accumulation, respectively See FIG. 2-3. While the extract again alleviated the tissue pathology. Along with these qualitative assays, serum and hepatic bio-chemical parameters, such as AST/ALT levels, TG and pro-inflammatory cytokine IL-6 levels also proved the inhibitory effect of the extract against ALD induced hepatosteatosis. See FIG. 4-8. In general, the beneficial effect of the above mentioned extract can be ranked in descending order (from the highest to the lowest) are as follows: Mixed extract-median dose>Mixed extract-high dose>Salvia miltiorrhiza roots extract>Hawthorn fruits extract=Mixed extract-low dose.

Experiment Example 1 data are summarized in Table 1 and in Table 2 below.

TABLE 1

| | | | Liver index (%) | AST (U/L) | ALT (U/L) |
|---|---|---|---|---|---|
| Control diet | | | 3.717 ± | 33.66 ± 2.516 *** | 8.289 ± 0.6629 *** |
| | | | 0.04889 *** | | |
| Ethanol | V | | 4.963 ± 0.1598 | 82.28 ± 4.915 | 40.66 ± 2.396 |
| diet | H-extract | | 4.322 ± 0.1091 *** | 49.59 ± 3.324 *** | 17.58 ± 2.535 *** |
| | S-extract | | 4.343 ± | 63.52 ± 3.134 ** | 23.32 ± 2.424 *** |
| | | | 0.09837 ** | | |
| | Mixed | L | 4.296 ± 0.1186 ** | 67.00 ± 3.960 * | 21.43 ± 1.795 *** |
| | extract | | | | |
| | | M | 4.146 ± 0.1200 ** | 41.61 ± | 15.01 ± 1.952 ***※ |
| | | | | 2.238 ***※※※ | |
| | | H | 4.137 ± | 45.22 ± | 16.07 ± 1.453 ***※ |
| | | | 0.07958 *** | 2.737 ***※※※ | |

TABLE 2

| | | | Serum TG | Hepatic TG | IL-6 (Relative gene |
|---|---|---|---|---|---|
| | | | (mmol/L) | (mmol/gprot) | expression folds) |
| Control diet | | | 0.7410 ± 0.04384 *** | 0.07441 ± 0.004314 *** | 1.059 ± 0.1652 ** |
| Ethanol | V | | 1.192 ± 0.05717 | 0.4918 ± 0.02404 | 5.099 ± 0.9188 |
| diet | H-extract | | 1.161 ± 0.04097 | 0.4492 ± 0.05521 | 1.528 ± 0.1803 ** |
| | S-extract | | 1.032 ± 0.03022 * | 0.3159 ± 0.02737 ** | 2.901 ± 0.5288 |
| | Mixed | L | 1.068 ± 0.05715 | 0.3651 ± 0.02980 *** | 1.714 ± 0.2268 ** |
| | extract | M | 0.9421 ± 0.05209 **## | 0.2310 ± 0.02089 ***##※ | 1.122 ± 0.2479 **※ |
| | | H | 1.013 ± 0.06411 | 0.2886 ± 0.02930 ***# | 1.320 ± 1.1875 **※ |

### Experiment Example 2

The inhibitory effect of mixed extract against ethanol diet induced Alcoholic liver fibrosis. Experiments were performed to examine the beneficial effects of mixed extract, as well as each compositions in treating ethanol diet induced mice liver fibrosis.

Extracts Preparation—

Done as in Experiment Example 1.

Experimental Groups—

Done as in Experiment Example 1.

Experimental Animal—

Done as in Experiment Example 1. In order to further investigate the therapeutic effect of the mixed extract towards alcoholic liver fibrosis. C57BL/6 male mice (8-9 weeks) were randomly selected for each group. The control group was fed with control liquid diet during the whole process. At beginning, pathological groups with or without extracts treatment (with same dosage as Experiment Example 1) were all fed with 1% (v/v) ethanol diet for 2 days and double the ethanol dosage on third and fourth day for adaption. Later on, these groups were then fed with 2% (v/v) ethanol diet for another 2 weeks with occasionally CCl4 injection (twice a week). Seventy two hours after the last injection, mice were sacrificed and the tissues were harvested for further evaluation. Note “v/v” means volume/volume.

Statistical analysis—as done in Experiment Example 1.

### Experiment Example 2 Results

At the end of model, mice suffered from alcoholic liver fibrosis and have elevated liver/body ratio. Both single extract and mixed extract dramatically alleviating ALD pathogenesis. This was also proved by microscopic observations. H&E staining demonstrated that ethanol diet with CCl4 injection easily triggered the proliferation of hepatic fibers. Most importantly, treating with a Hawthorn fruits extract, treating with a Salvia miltiorrhiza roots extract, and treating with the mixed extract of Hawthorn fruits and Salvia miltiorrhiza roots all significantly improved the liver tissue pathology. Also, the Masson's trichrome stain and the ORO staining shared similar trends of improvements and reductions in FLD. Also it is important to note that the beneficial effect of the above mentioned extract can be ranked (from the best effective treatment to least effective treatment. The ranking of the extracts was as follows: Mixed extract-median dose=Mixed extract-high dose>Salvia miltiorrhiza roots extract>Hawthorn fruits extract—Mixed extract-low dose.

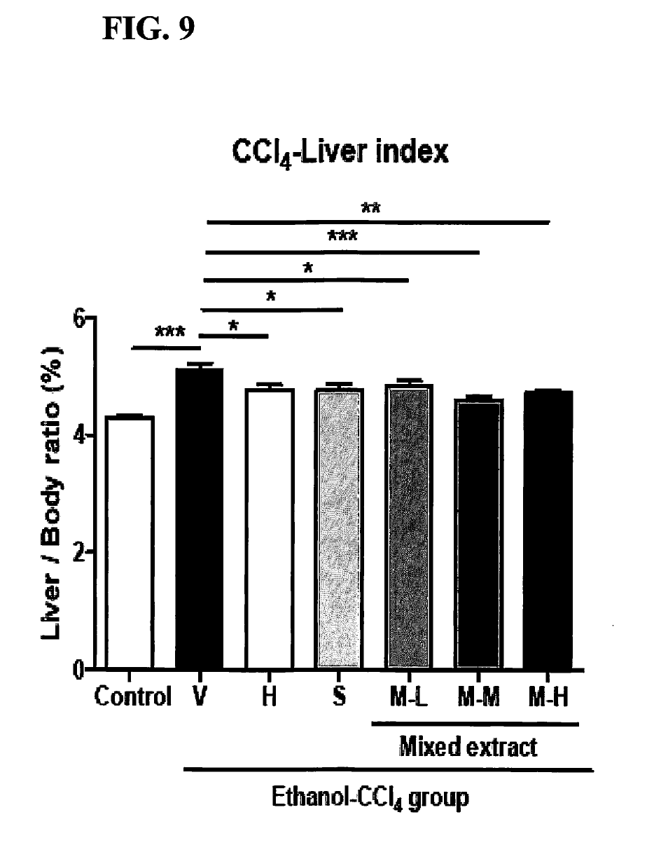

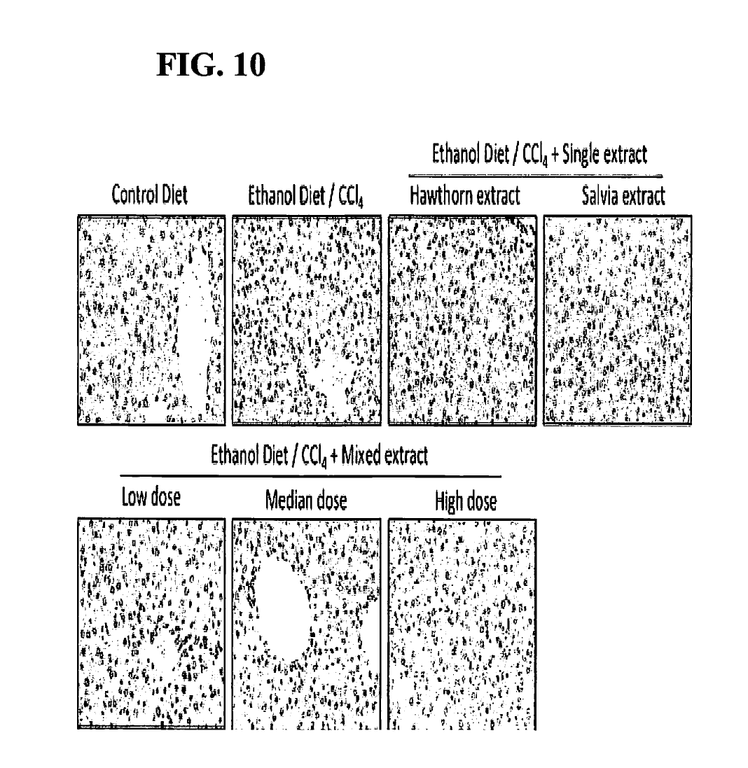

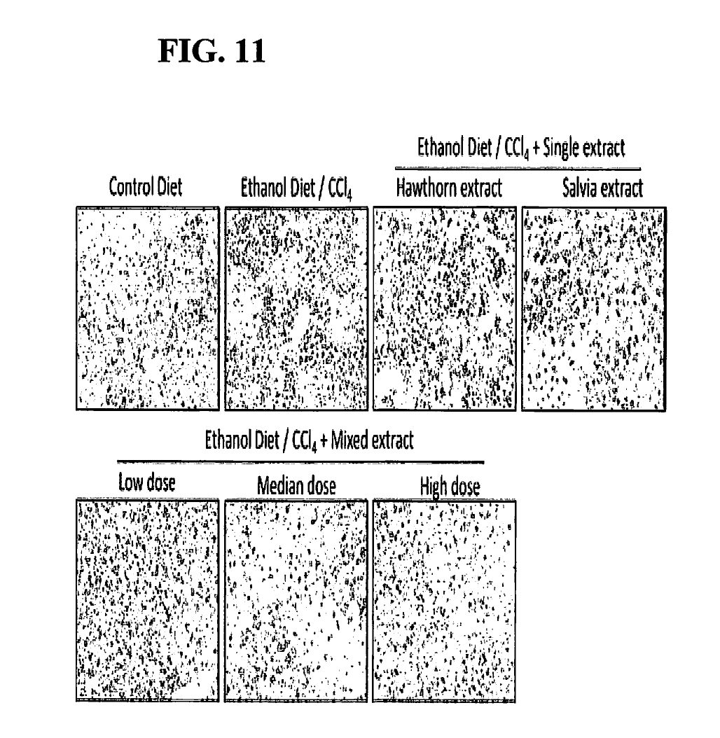

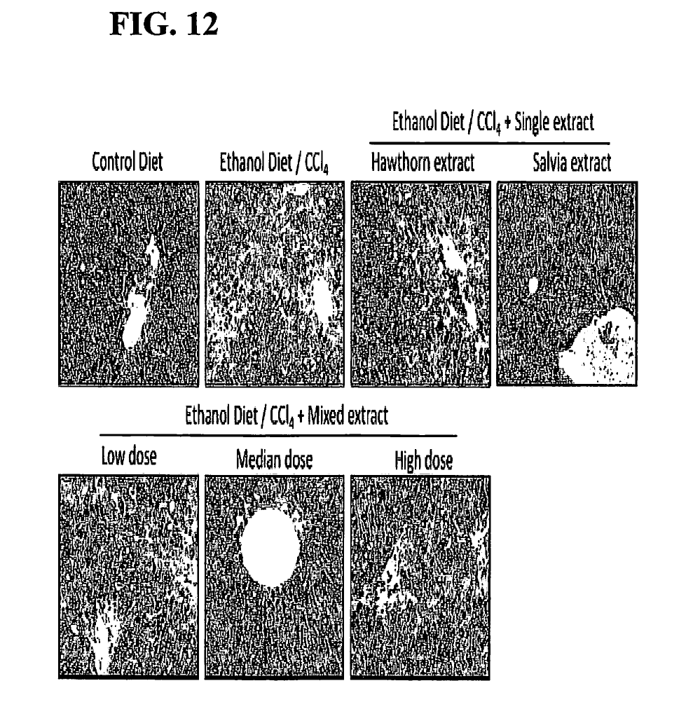

Experiment Example 2 data are summarized in Table 3 below. (See also FIGS. 9-12)

TABLE 3

| | | | Liver index (%) |
|---|---|---|---|
| Control diet | | | 4.298 ± 0.03916 *** |
| Ethanol | V | | 5.132 ± 0.09343 |
| diet with | H-extract | | 4.778 ± 0.08548 * |
| CCl₄ | S-extract | | 4.763 ± 0.1133 * |
| injection | Mixed | L | 4.834 ± 0.09731 * |
| | extract | M | 4.610 ± 0.07058 *** |
| | | H | 4.738 ± 0.04565 ** |

### Experiment Example 3

The inhibitory effect of mixed extract against high fat diet induced obesity. Experiments were performed to examine the beneficial effects of mixed extract, as well as each compositions in preventing high fat diet induced mice weight gain.

Extracts preparation—as done in Experiment Example 1, while the weight ratio in mixed extract was further optimized as 1:1 (Hawthorn fruits flavonoid:tanshinone). Again, for convenient purpose, the dosages mentioned below represent flavonoid contents in Hawthorn fruits extract, or tanshinone contents in Salvia miltiorrhiza roots extract. Which were commonly accepted most active ingredients in the ethanol extract of both herbs.

Experimental animal—as done in Experiment Example 1. In obesity and NAFLD model, 6-7 weeks old C57BL/6 female mice were fed with either control diet or high fat diet for 10 weeks. Briefly, mice were randomly into six groups, with 5-6 mice per group. These groups were then challenged with different diet and extracts treatments. For control group, mice were fed with normal diet (named as ND). For obesity/NAFLD group, mice were fed with high fat diet. To investigate the beneficial effect of each extract towards fatty liver disease, extracts were administrated daily by intragastrical method. Mice treated with 100 mg/kg single extract of Hawthorn fruits or Salvia milliorrhiza roots were labeled as H and S, respectively. Additionally, mice were orally gavaged with mixed extract at low (50-50 mg/kg) or high dosage (100-100 mg/kg); which were labeled as M-L and M-H, respectively. At the end of model, mice were sacrificed and the tissues were collected for further investigation.

Statistical analysis—as done in Experiment Example 1.

Results—

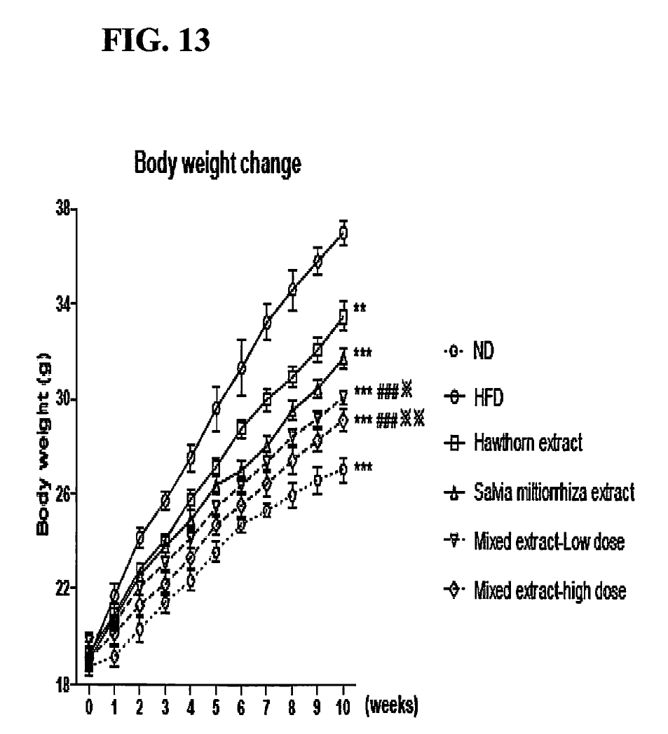

At the end of model, mice fed with HFD had excessive weight gain comparing with their normal diet counterparts (See FIG. 13). Single extract from either Hawthorn fruits or Salvia miltiorrhiza roots showed sufficient inhibitory effect towards the hasty weight gain. Which can be further optimized after mixing the extracts together. The beneficial effect of the treatments towards mice obesity can be ranked in descending order (from the highest to the lowest) are as follows: Mixed extract-high dose>Mixed extract-low dose>Salvia miltiorrhiza roots extract>Hawthorn fruits extract.

Experiment Example 3 data are summarized in Table 4 below.

TABLE 4

| | | | Body weight (g) |
|---|---|---|---|
| Control diet | | | 27.92 ± 0.5286 *** |
| High | V | | 36.98 ± 0.4879 |
| fat diet | H-extract | | 33.45 ± 0.6103 ** |
| | S-extract | | 31.71 ± 0.4483 *** |
| | Mixed | L | 30.06 ± 0.2970 ***###※ |
| | extract | H | 29.08 ± 0.4913 ***###※※ |

### Experiment Example 4

The inhibitory effect of mixed extract against high fat diet induced NAFLD. Experiments were performed to examine the beneficial effects of mixed extract, as well as each compositions in treating high fat diet induced NANFLD.

Extracts preparation—same as Experiment Example 3.

Experimental animal—same as Experiment Example 3.

Statistical analysis—same as Experiment Example 3.

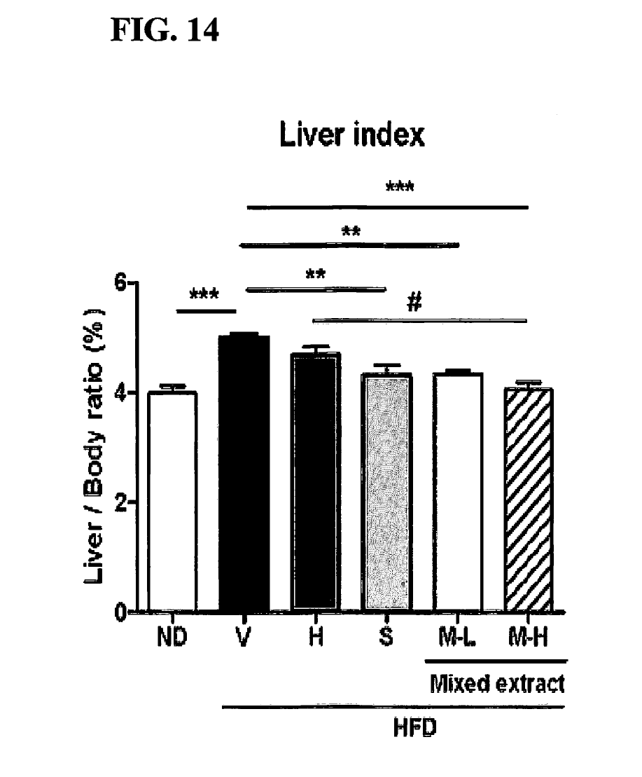

Results—as results, the beneficial effect of the extracts towards NAFLD can be ranked in the same descending order as Experiment Example 3: mixed extract-high dose>mixed extract-low dose>salvia miltiorrhiza roots extract>hawthorn fruits extract. The detail data is discussed below in Tables 5, 6, and 7. Briefly, mice challenged with HFD had a wide range of abnormal hepatic parameters. Which included liver enlargement and excessive induction of liver—body weight ratio (FIG. 14).

Experiment Example 4 data are summarized in Tables 5, 6 and 7 below.

TABLE 5

| | | | liver index (%) | AST (U/L) |
|---|---|---|---|---|
| Control diet | | | 3.999 ± 0.1286 *** | 54.80 ± 2.267 *** |
| High fat | V | | 5.028 ± 0.06149 | 138.2 ± 11.52 |
| diet | H-extract | | 4.696 ± 0.1492 | 80.50 ± 12.98 ** |
| | S-extract | | 4.330 ± 0.1736 ** | 105.7 ± 15.34 |
| | Mixed | L | 4.341 ± 0.05991 ** | 89.17 ± 14.55 * |
| | extract | H | 4.069 ± 0.1252 ***# | 70.80 ± 7.074 ** |

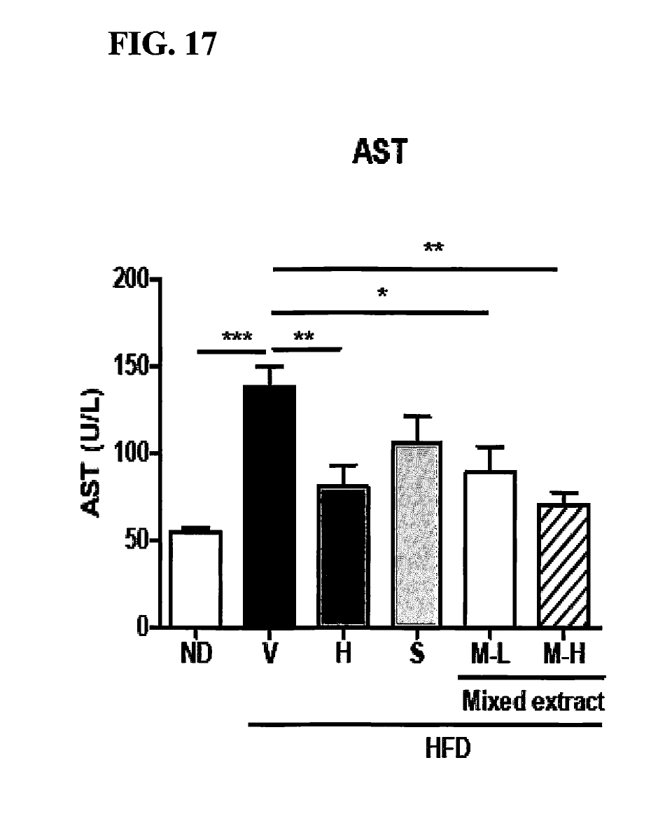

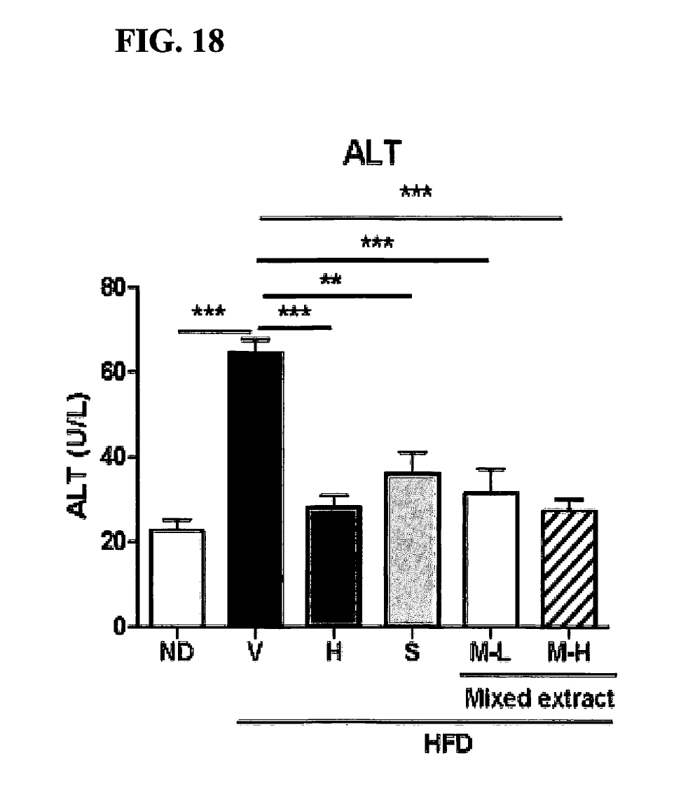

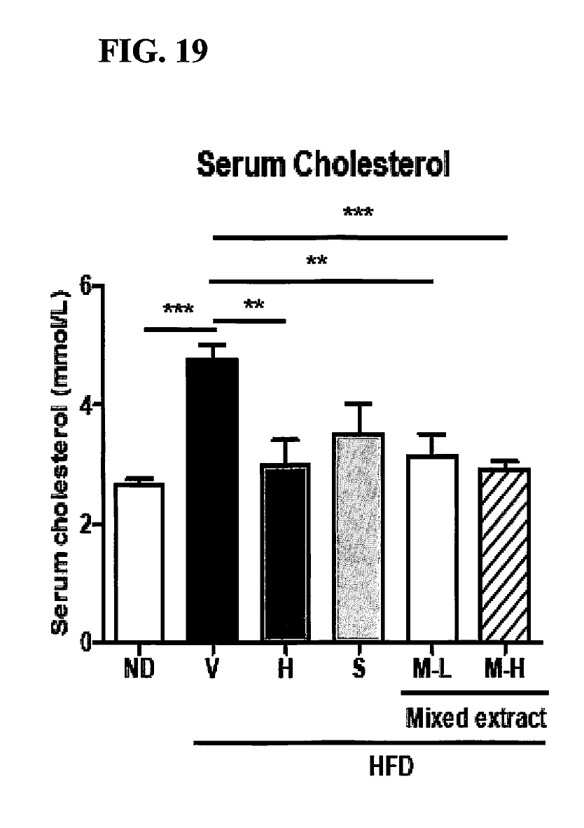

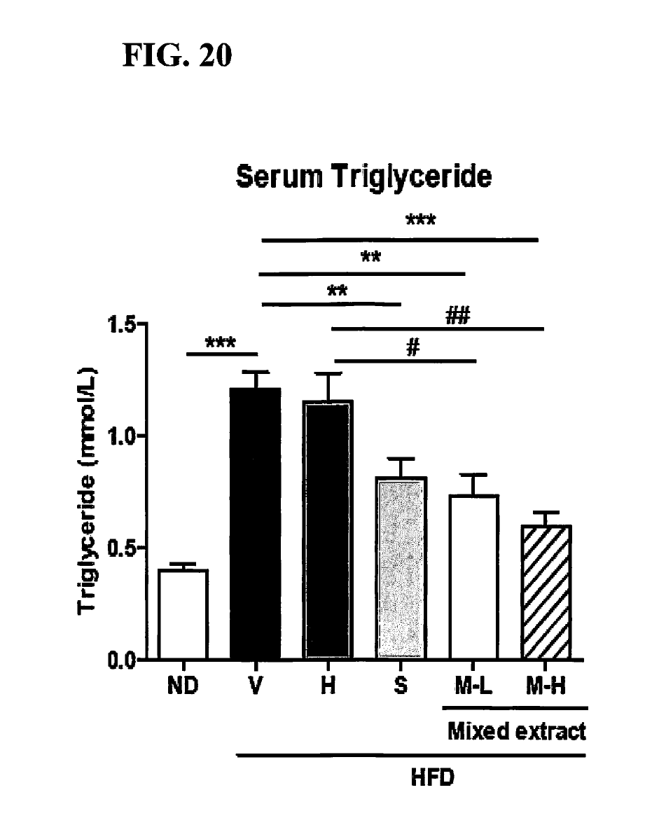

Along with the abnormal liver index, serum biochemical parameters such AST (see Table 5 above), ALT, TC (see Table 6 below) and TG levels (see Table 7 below) demonstrate liver injury and lipid accumulations during the feeding period (See FIG. 17-20).

TABLE 6

| | | | ALT (U/L) | TC (mmol/L) |
|---|---|---|---|---|
| Control diet | | | 22.60 ± 2.581 *** | 2.664 ± 0.09431 *** |
| High fat | V | | 64.60 ± 3.140 | 4.758 ± 0.2738 |
| diet | H-extract | | 28.17 ± 2.713 *** | 3.000 ± 0.4126 ** |
| | S-extract | | 36.17 ± 5.141 ** | 3.517 ± 0.4846 |
| | Mixed | L | 31.67 ± 5.631 *** | 3.155 ± 0.3554 ** |
| | extract | H | 27.20 ± 2.818 *** | 2.907 ± 0.1460 *** |

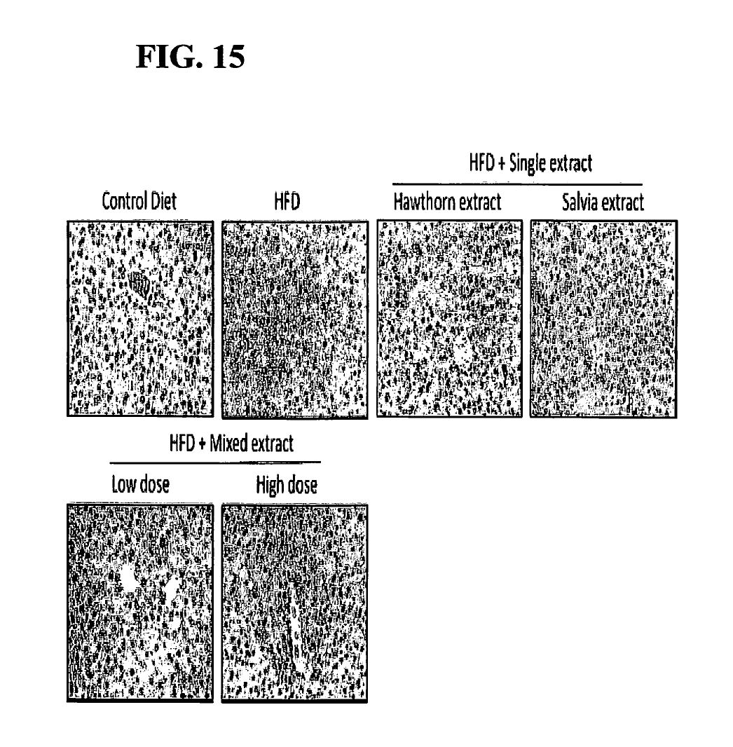

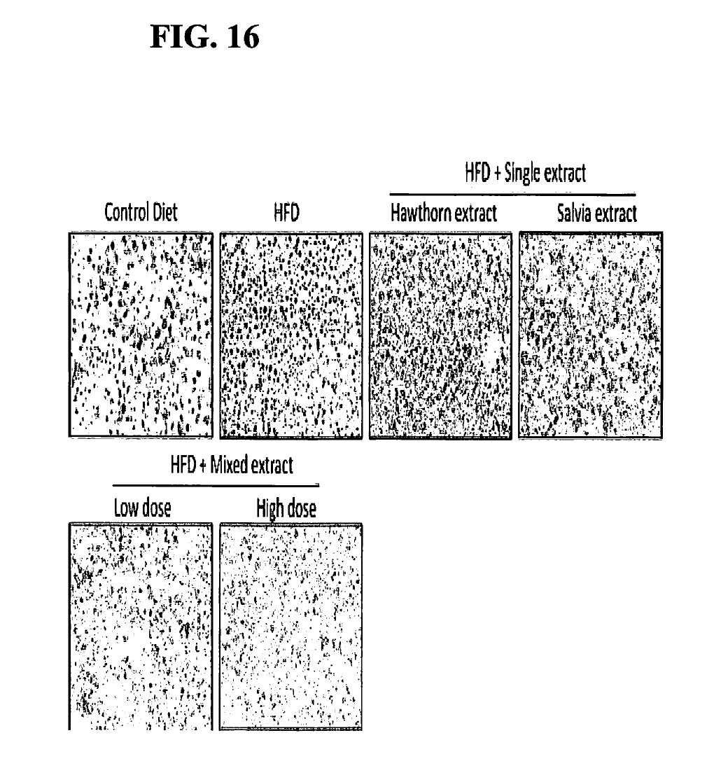

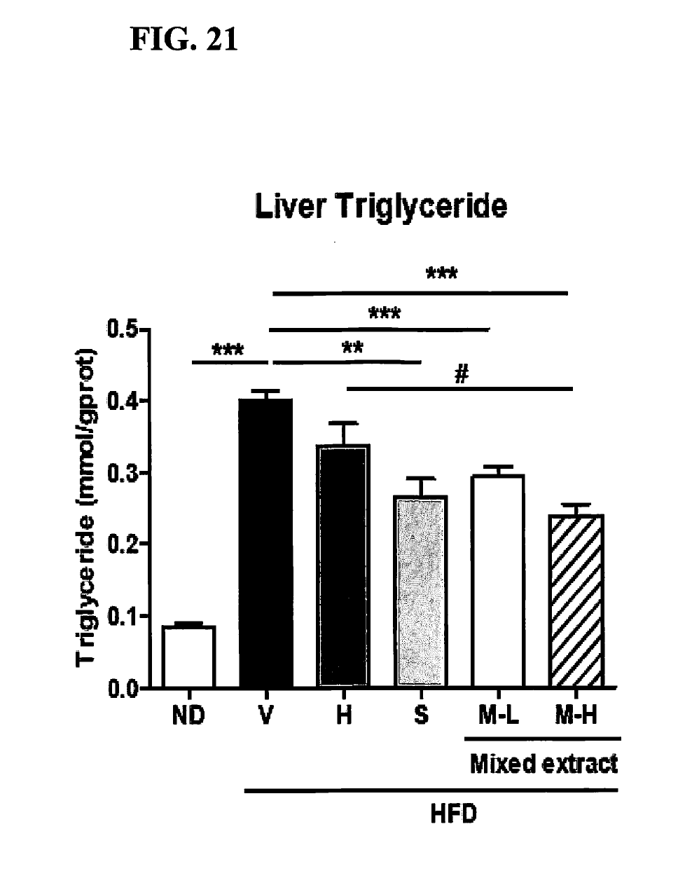

Moreover, histological staining observed similar pathological features in the livers of HFD mice as ALD group, such as balloon-like lesion and big lipid droplets as shown in FIG. 15-16. Also, for confirmation by quantitative hepatic TG level analysis, see FIG. 21.

TABLE 7

| | | | | Hepatic |
|---|---|---|---|---|
| | | | Serum TG (mmol/L) | TG (mmol/gprot) |
| Control diet | | | 0.3998 ± 0.02870 *** | 0.08550 ± 0.005695 *** |
| High fat | V | | 1.908 ± 0.07584 | 0.4010 ± 0.01263 |
| diet | H-extract | | 1.152 ± 0.1300 | 0.3378 ± 0.03034 |
| | S-extract | | 0.8131 ± 0.08772 ** | 0.2644 ± 0.02674 ** |
| | Mixed | L | 0.7341 ± 0.09462 **# | 0.2932 ± 0.01471 *** |
| | extract | H | 0.5940 ± 0.06284 ***## | 0.2390 ± 0.01656 ***# |

Similar as their performance in ALD model, both Hawthorn fruits extract and Salvia miltiorrhiza roots extract exhibited sufficient beneficial effect when they were administrated alone. With even stronger inhibitory effect of mixed extract, the abnormal liver index, tissue lesion and over-induced biochemical levels were significantly improved.

According to EXPERIMENT EXAMPLES 1-4, the mixed extract can be further developed into pharmaceutical composition, dietary supplement or functional food composition. In human cases, the mixed extract can be administered at daily dosage preferably from 5 mg/kg to 150 mg/kg, in once or a few divided doses, as necessary. Below, EXAMPLE 1-2 will reveal most potential utilities in detail.

### Example 1

Example 1 concerns a preparation of pharmaceutical composition or dietary supplement for prevention, treatment or amelioration of fatty liver disease and preferably the pharmaceutical composition or the dietary supplement are administered in the form of an oral administration selected from the group consisting of a powder, granules, atablet, a capsule, a suspension, an emulsion, a syrup, and an aerosol, and any combination thereof.
1) For example, making a powder, Hawthorn fruits extract and Salvia miltiorrhiza roots extract may be mixed thoroughly at weight ratio from 2:1 to 1:2. Some adjuvant materials such as acceptable pharmaceutical and food additives can be added.
2) For example, for making a tablet, Hawthorn fruits extract and Salvia miltiorrhiza roots extract may be mixed thoroughly at weight ratio from 2:1 to 1:2. After optionally screening, the mixed extract can be further made into tablets either by molding or compression, with or without suitable excipients known in tablet making.
3) For example, for making a capsule, Hawthorn fruits extract and Salvia miltiorrhiza roots extract can be mixed thoroughly at weight ratio from 2:1 to 1:2. After optionally screening, the mixed extract are dissolved or suspended into oil and further made into soft-shelled capsules. Alternatively, the mixed extract can be added into hard-shelled capsules with or without suitable pharmaceutical and food additives known in capsule making.
4) For making granules, suspensions, emulsions, syrups or aerosols, Hawthorn fruits extract and Salvia miltiorrhiza roots extract can be mixed thoroughly at weight ratio from 2:1 to 1:2. The mixed extracts are used in these dosage forms with typical excipients for these dosage forms and are manufactured under FDA guidelines for these dosage forms.

### Example 2

Preparation of functional food composition for benefiting fatty liver disease, would include beverage compositions of Hawthorn fruits extract and Salvia miltiorrhiza roots extract which have been mixed thoroughly at weight ratio from 2:1 to 1:2. After screening (optional), the powdered extract is dissolved or suspended in a beverage with or without alcohol. Some other food additives and follow-up processes may included such as a sweetener, sugar substitute, vitamin or CO₂ carbonation of the beverage step. Preferably, the weight ratio of mixed extract in final product ranges from 0.1 wt % to 12 wt %. Note “wt.” means weight.

Certain terminology may be used in the following description for convenience only and is not limiting. Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a tip” includes a plurality of tips. Thus, for example, a reference to “a method” includes one or more methods, and/or steps of the type described herein and/or which will become apparent to those persons skilled in the art upon reading this disclosure. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method for preventing a fatty liver disease in a person, the method comprising:
administering to the person a therapeutic composition selected from the group consisting of a pharmaceutical composition, a dietary supplement, a health functional food composition, and any combination thereof,
wherein the therapeutic composition comprises an active ingredient which comprises a mixture of pharmaceutically acceptable solvent extracts of Hawthorn fruits and Salvia miltiorrhiza roots; and
using the therapeutic composition administered to the person for preventing the fatty liver disease in the person.

2. The method according to claim 1, wherein the mixture of the pharmaceutically acceptable solvent extracts of the Hawthorn fruits and the Salvia miltiorrhiza roots is ethyl alcohol solvent extracts of the Hawthorn fruits and the Salvia miltiorrhiza roots.

3. The method according to claim 1, wherein the pharmaceutically acceptable solvent extracts of the Hawthorn fruits and the Salvia miltiorrhiza roots are ethyl alcohol in water solvent extracts of the Hawthorn fruits and the Salvia miltiorrhiza roots.

4. The method according to claim 1, wherein the fatty liver disease is selected from the group consisting of a non-alcoholic fatty liver disease, and an alcoholic liver disease.

5. The method according to claim 2, wherein the mixture of the ethyl alcohol solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

6. The method according to claim 3, wherein the mixture of the ethyl alcohol in the water solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

7. The method according to claim 1,
wherein the therapeutic composition further comprises an excipient ingredient selected from the group consisting of a pharmaceutically acceptable carrier, a purified water, an isotonic saline, a surfactant, a flavoring agent, a colorant, a diluent, a disintegrant, a sugar, a protein, a fat, a gum, and any combination thereof,
wherein the administering to the person of the therapeutic composition occurs by a route of administration selected from the group consisting of an oral route, a parenteral route, an intravenous route, a subcutaneous route, an intraperitoneal route, a topical application route, a transdermal patch route, a nasal spray route, a pulmonary aerosol route, a suppository route, and any combination thereof, and
wherein a total daily dosage of the mixture of pharmaceutically acceptable solvent extracts of the Hawthorn fruits and the Salvia miltiorrhiza roots as calculated per kilogram body weight of the person is the total daily dosage selected from the group consisting of between about 1 mg to about 10 mg, between about 10 mg to about 40 mg, between about 40 mg to about 70 mg, between about 70 mg to about 100 mg, between about 100 mg to about 130 mg, between about 130 mg to about 160 mg, between about 160 mg to about 190 mg, between about 190 mg to about 210 mg, between about 210 mg to about 240 mg, between about 240 mg to about 270 mg, between about 270 mg to about 300 mg, between about 300 mg to about 330 mg, between about 330 mg to about 360 mg, between about 360 mg to about 390 mg, between about 390 mg to about 420 mg, and between about 420 mg to about 450 mg, and
wherein the total daily dosage of the mixture of pharmaceutically acceptable solvent extracts of the Hawthorn fruits and the Salvia miltiorrhiza roots is administered in one dose per day or in divided doses each day.

8. A method for treating a fatty liver disease in a person, the method comprising:
administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and
using the therapeutic composition administered to the person for treating the fatty liver disease in the person.

9. The method according to claim 8, wherein the mixture of the pharmaceutically acceptable solvent extracts of the Hawthorn fruits and the Salvia miltiorrhiza roots are ethyl alcohol solvent extracts of the Hawthorn fruits and the Salvia miltiorrhiza roots.

10. The method according to claim 8, wherein the mixture of the pharmaceutically acceptable solvent extracts of the Hawthorn fruits and the Salvia miltiorrhiza roots is ethyl alcohol in water solvent extracts of the Hawthorn fruits and the Salvia miltiorrhiza roots.

11. The method according to claim 8, wherein the fatty liver disease is selected from the group consisting of a non-alcoholic fatty liver disease, and an alcoholic fatty liver disease.

12. The method according to claim 9, wherein the mixture of the ethyl alcohol solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

13. The method according to claim 10, wherein the mixture of the ethyl alcohol in the water solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, and between about 10 to about 50.

14. The method according to claim 8,
wherein the therapeutic composition is the health functional food composition,
wherein the mixture of the pharmaceutically acceptable solvent extracts of the Hawthorn fruits and the Salvia miltiorrhiza roots is added to the therapeutic health food composition, and
wherein the mixture of the pharmaceutically acceptable solvent extracts of the Hawthorn fruits and the Salvia miltiorrhiza roots has a weight which is between about 0.1 percent to about 12 percent of the weight of the therapeutic health food composition.

15. The method according to claim 8,
wherein the therapeutic composition further comprises an excipient ingredient selected from the group consisting of a pharmaceutically acceptable carrier, a purified water, an isotonic saline, a surfactant, a flavoring agent, a colorant, a diluent, a disintegrant, a sugar, a protein, a fat, a gum, and any combination thereof,
wherein the therapeutic composition is administered by a route of administration selected from the group consisting of an oral route, a parenteral route, an intravenous route, a subcutaneous route, an intraperitoneal route, a topical application, a transdermal patch route, a nasal spray route, a pulmonary aerosol route, a rectal suppository route, and any combination thereof, and
wherein a total daily dosage of the mixture of pharmaceutically acceptable solvent extracts of the Hawthorn fruits and the Salvia miltiorrhiza roots as calculated per kilogram body weight of the person is selected from the group consisting of between about 1 mg to about 10 mg, between about 10 mg to about 40 mg, between about 40 mg to about 70 mg, between about 70 mg to about 100 mg, between about 100 mg to about 130 mg, between about 130 mg to about 160 mg, between about 160 mg to about 190 mg, between about 190 mg to about 210 mg, between about 210 mg to about 240 mg, between about 240 mg to about 270 mg, between about 270 mg to about 300 mg, between about 300 mg to about 330 mg, between about 330 mg to about 360 mg, between about 360 mg to about 390 mg, between about 390 mg to about 420 mg, and between about 420 mg to about 450 mg, and
wherein the total daily dosage of the mixture of pharmaceutically acceptable solvent extracts of the Hawthorn fruits and the Salvia miltiorrhiza roots is administered in one dose a day, or in divided doses during the day.

16. A method for ameliorating a fatty liver disease in a person, the method comprising:
administering to the person a therapeutic composition selected from the group consisting of a pharmaceutical composition, a dietary supplement, a health functional food composition, and any combination thereof, wherein the therapeutic composition comprises an active ingredient which comprises a mixture of pharmaceutically acceptable solvent extracts of Hawthorn fruits and Salvia miltiorrhiza roots; and
using the therapeutic composition administered to the person for ameliorating the fatty liver disease in the person.

17. The method according to claim 16, wherein the mixture of the pharmaceutically acceptable solvent extracts of the Hawthorn fruits and the Salvia miltiorrhiza roots are ethyl alcohol solvent extracts of the Hawthorn fruits and the Salvia miltiorrhiza roots.

18. The method according to claim 16, wherein the mixture of the pharmaceutically acceptable solvent extracts of the Hawthorn fruits and the Salvia miltiorrhiza roots are ethyl alcohol in water solvent extracts of the Hawthorn fruits and the Salvia miltiorrhiza roots.

19. The method according to claim 16 wherein the mixture of the pharmaceutically acceptable solvent extracts of the Hawthorn fruits and the Salvia miltiorrhiza roots is in a mixture ratio selected from the group consisting of between about 0.02 to about 0.1, between about 0.1 to about 0.2, between about 0.2 to about 0.3, between about 0.3 to about 0.4, between about 0.4 to about 0.5, between about 0.6 to about 0.7, between about 0.8 to about 0.9, between about 0.9 to about 1.0, 1.0, between about 1.0 to about 1.1, between about 1.1 to about 1.3, between about 1.3 to about 1.5, between about 1.5 to about 1.7, between about 1.7 to about 2.0, between about 2.0 to about 2.5, between about 2.5 to about 3.3, between about 3.3 to about 4, between about 4 to about 6, between about 6 to about 10, between about 10 to about 50.

20. The method according to 15, wherein the fatty liver disease is selected from the group consisting of an alcoholic liver disease, and a non-alcoholic fatty liver disease.
